(19) United States
(12) Patent Application Publication
(10) Pub. No.: US 2025/0002488 A1
(43) Pub. Date: Jan. 2, 2025

(54) Glioblastoma Multiforme Treatments

(71) Applicants: Brown University, Providence, RI (US); Rhode Island Hospital, Providence, RI (US)

(72) Inventors: Nikolaos Tapinos, Barrington, RI (US); Ola Hassan, Worcester, MA (US); David Karambizi, Providence, RI (US)

(21) Appl. No.: 18/700,506

(22) PCT Filed: Oct. 11, 2022
(86) PCT No.: PCT/US2022/077910

§ 371 Date: Apr. 11, 2024

Related U.S. Application Data
(60) Provisional application No. 63/254,826, filed on Oct. 12, 2021.

(51) Int. Cl. C07D 417/14; A61K 31/165; A61K 31/167; A61K 31/4245; A61P 35/00; C12N 15/113

(52) U.S. Cl. CPC C07D 417/14; A61K 31/165; A61K 31/167; A61K 31/4245; A61P 35/00; C12N 15/1137; C12N 2310/14

(57) ABSTRACT

HDAC-7 inhibitors and uses thereof are provided, including their uses as therapeutic agents for treating brain cancer and/or solid tumors, for example, glioblastoma multiforme (GBM). In some embodiments, provided herein are HDAC-7 inhibitors selected from small molecules, siRNAs, or HDAC class IIa inhibitors, such as TMP269, or pharmaceutically acceptable salts thereof.

### RELATED APPLICATIONS

This application claims the benefit of U.S. Provisional patent application 63/254,826 filed Oct. 12, 2021, the entire contents of which are incorporated by reference herein.

### SEQUENCE LISTING

This application contains a sequence listing having the filename GLIOBLASTOMA MULTIFORME TREATMENT.xml, which is 4,589 bytes in size, and was created on Sep. 26, 2022. The entire contents of this sequence listing are incorporated by reference herein.

### FIELD

The present disclosure relates to the treatment of brain cancer and/or solid tumors, for example, glioblastoma multiforme (GBM) using inhibitors of histone deacetylase 7 (HDAC-7).

### BACKGROUND

Glioblastoma multiforme (GBM) is the most malignant and aggressive primary brain tumor. GBM has an ominous prognosis with a survival rate of 14-15 months after diagnosis. Despite worldwide initiatives to optimize therapeutic approaches, GBM is still among the most challenging diseases to treat and the fastest to relapse in clinical oncology. Treatment resistance of GBM and the inevitable tumor recurrence are primarily attributed to the presence of tumor-initiating cells or glioma stem cells (GSCs).

Because of the foregoing shortcomings, there is an urgent need for an effective GBM treatment.

### SUMMARY

Inhibitors of Histone Deacetylase 7 (HDAC-7) were chosen as a potential therapeutic target based upon the discovery that HDAC-7 is highly expressed in glioblastoma compared to normal brain tissue, and the discoveries that inhibiting HDAC-7 has a significant effect on GBM growth, and inhibits GSC properties, transcriptomic expression, and invasion of cancer cells.

A small molecule inhibitor library that shows significant specificity towards HDAC-7 compared to other Class IIa Histone Deacetylases was designed and screened to determine possible drugs against human brain and other solid tumors. These small molecules, or pharmaceutically acceptable salts thereof, are disclosed herein.

In addition to the small molecules, disclosed herein are inhibitors selected from the SIRNAS GACAAGAGCAAGCGAAGUG (SEQ ID NO:1), GCAGAUACCCUCGGCUGAA (SEQ ID NO: 2), GGUGAGGGCUUCAAUGUCA (SEQ ID NO:3), or UGGCUGCUCUUCUGGGUAA (SEQ ID NO: 4). Other HDAC-7 inhibitors include TMP269, trichostatin A, and vorinostat.

These HDAC-7 inhibitors, or pharmaceutically acceptable salts thereof, are useful in treating brain cancer and/or solid tumors, for example, glioblastoma multiforme (GBM).

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 shows a graphical abstraction of one possible histone HDAC-7 inhibition model approach as a GBM therapy, including a schematic of the unraveled HDAC-7 interactome and a schematic of the posttranslational modification after HDAC-7 inhibition in order to inhibit certain cell cycle processes and cell division signaling to regulate the self-renewal of GSCs.

FIGS. 2A-2F show a 5-gene epigenetic signature for IDH wildtype glioblastoma. Overview of the in-vivo pooled overexpression epigenetic screen (FIG. 2A). Genes that differentially represented in the tumor versus baseline control (FIG. 2B). Selection of clinically relevant genes across 3 GBM cohorts (FIG. 2C). Waterfall plot showing similar cut-off from TCGA being applied to Gravendeel cohort (FIG. 2D). Overall survival analysis of high risk (HR) and low risk (LR) subgroups stratified using the 5-gene signature in the training and validation sets (FIG. 2E). Subclass mapping analysis of the HR and LR subgroups (SubMap software). Significant p-value confirmed that HR subgroup of TCGA cohort had significant correspondence with HR subgroup of Gravendeel cohort; the same was for the LR (FIG. 2F).

FIGS. 3A-3F. Summary of the NanoString data. Overview of NanoString analysis of 84 GBM patient samples (FIG. 3A). Overall survival (FIG. 3B) and progression-free survival (FIG. 3C) analyses of the HR versus LR subgroups stratified with the 5-gene signature. Heatmap showing genes that are differentially expressed in the HR versus LR tumors (FIG. 3D). DAVID analysis of the differentially expressed genes in the HR/LR tumors (FIG. 3E). Volcano plot representation of differential expression analysis of genes expressed in the HR/LR tumors (FIG. 3F).

FIGS. 4A-4B show HDAC-7 expression in the Chinese Glioma Genome Atlas (CGGA). The expression of HDAC-7 in the different forms of the glial tumors for patients from the CGGA data with GBM showing the highest HDAC-7 expression (FIG. 4A). Kaplan-Meyer Survival analysis for all the GBM patients from CGGA data are divided into two groups (“high HDAC-7 expression” and “low HDAC-7 expression”) with HDAC-7 “high expression” group showing less survival compared to HDAC-7 “low expression” group (FIG. 4B).

FIGS. 5A-5D show HDAC-7 expression in The Cancer Genome Atlas (CGGA). The expression of HDAC-7 in the GBM tumors versus non-tumors from the TCGA data with GBM showing a pronounced high HDAC-7 expression relative to non-tumors (FIG. 5A). Kaplan-Meyer Survival Analysis for all the GBM patients from TCGA data are divided into two groups (“high HDAC-7 expression” and “low HDAC-7 expression”) with HDAC-7 “high expression” risk patient group shows less survival compared to “low expression” risk patient (FIG. 5B). Heat Map representation and Volcano plot representation respectively of differential expression analysis of genes expressed in the HDAC-7 high expression group versus the HDAC-7 low expression group in the GBM cohorts from TCGA data (FIGS. 5C-5D).

FIGS. 6A-6D show GSCs treated with HDAC class IIa inhibitor are unable to form neuro-spheres in a 2-week limiting dilution assay. Images for GSC cultures treated with DMSO (control) and HDAC class IIa inhibitor respectively in a limiting dilution assay ‘LDA’, where GSCs treated with the HDAC class IIa inhibitor are unable to form neuro-spheres in a 2-week LDA compared to the control GSCs that showed sphere forming ability at a very early time point (FIGS. 6A and 6C). A graph using an online algorism to score and assess the self-renew ability of GSCs treated with DMSO and HDAC class IIa inhibitor respectively (FIGS. 6B and 6D).

FIGS. 7A-7D show transcriptomic analysis for HDAC-7 siRNA. Heat Map representation and Volcano plot representation respectively of the differentially expressed genes in the HDAC-7 knockdown versus control from RNASeq applied after siRNA knocking down in primary GSCs (FIGS. 7A-7B). Gene enrichment analysis of the differentially expressed genes in the HDAC-7 knockdown versus control from RNASeq applied after siRNA knocking down in primary GSCs (FIG. 7C). Overlap radar chart displaying the overlap between the input downregulated genes among the different stem cell types (FIG. 7D).

FIGS. 8A-8B show HDAC-7 inhibition suppresses each of mesenchymal and proneural phenotypes for GSCs. GSEA enrichment plots of HDAC-7-knocked down GSCs versus control using two different signature gene sets showing that HDAC-7 inhibition inhibits the signature genes of the mesenchymal and the pnoneural phenotypes (FIG. 8A). The normalized enrichment scores (NES) are shown for each plot. HDAC-7 expression is relatively close in the different GSCs' subtypes form TCGA publicly available data (FIG. 8B).

FIGS. 9A-9B show HDAC-7 relative protein levels in WT GSCs and after siRNA knockdown. The relatively low protein abundance of HDAC-7 in GSCs as compared to a housekeeping gene (FIG. 9A). The relative protein levels of HDAC-7 in GSCs after HDAC-7 SIRNA vs control (FIG. 9B).

FIG. 10 shows HDAC-7 inhibition leads to specific changes to histones PTM in GSCs. Heat Map representation of the changes in the abundance of histones PTMs in the HDAC-7 knockdown versus control detected through Mod Spec. Experiment ran three independent runs as technical replicates.

FIG. 11 shows HDAC-7 inhibition leads to specific changes to other HDAC members in GSCs. Plots show normalized counts from RNASeq data for the effect on other HDACs in GSCs following knocking down HDAC-7 using siRNA versus control, P-adjust <0.05.

FIG. 12 shows Class IIa HDAC inhibitors with comparable inhibitory effect on the cell viability of GSCs. Dose response curve for GSCs treated with Pan HDAC inhibitors TSA (Trichostatin A), SAHA (Vorinostat) versus the HDAC class IIa inhibitor (TMP269). Cell viability assay was determined by MTS after the treatment with increasing doses of each corresponding drug at time point 72 hrs with P-value <0.0001.

FIGS. 13A-13B. Kaplan-Meier Survival Analysis for GBM patients from RIH. Data are divided into two groups with HDAC-7 “high expression” patient group showing less survival compared to “low expression” patients (FIG. 13A). The expression of HDAC-7 in GBM tumors versus non-tumors from the TCGA with GBM showing a high HDAC-7 expression relative to non-tumors (FIG. 13B).

FIGS. 14A-14E. Transcriptomic analysis for HDAC-7 siRNA. Heat Map (FIG. 14A) and Volcano plot representation (FIG. 14B) of 4963 differentially expressed genes in the HDAC-7 siRNA knockdown versus control in primary GSCs. Gene Ontology enrichment analysis of the differentially expressed genes in the HDAC-7 knockdown versus control (FIG. 14C). Checkerboard table shows the occurrence of each individual knocked down gene and its occurrence across different stemness signature data bases (FIG. 14D). Radar chart displaying the overlap between the si-HDAC-7 downregulated genes among different stem cell types (FIG. 14E).

FIGS. 15A-15B. Proteomic analysis for HDAC-7 siRNA. Heat Map representation of the changes in the abundance of histones posttranslational modification in the HDAC-7 knockdown versus control detected through Mod Spec (Mass Spec) (FIG. 15A). Schematic representation for the DNA binding proteins and chromatin associated protein partners with HDAC-7 identified through RIME (FIG. 15B). The identified proteins were in close-proximity (2.3-2.7 Å) to HDAC-7 in the GSCs.

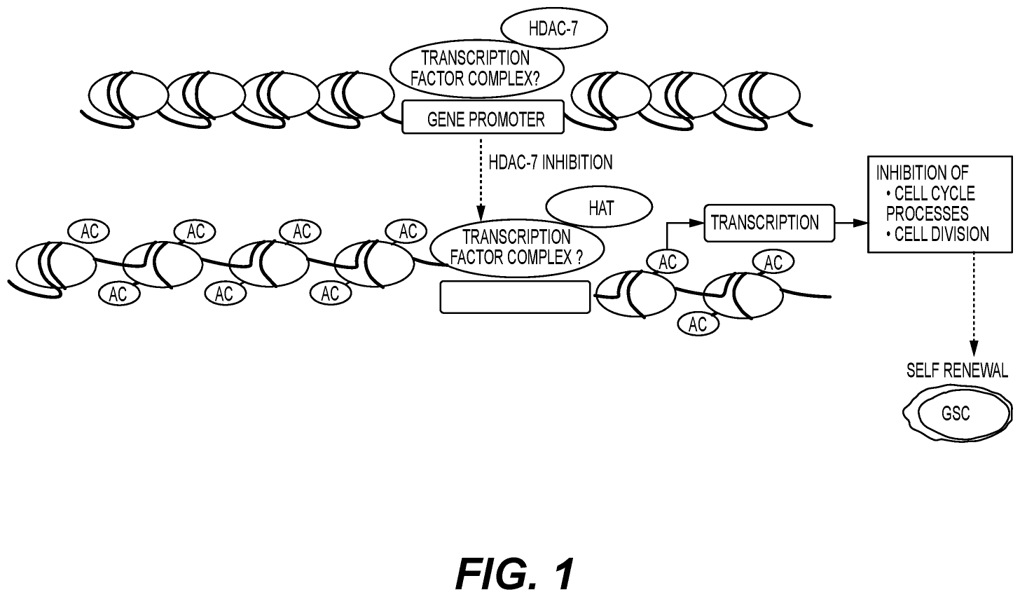

### DETAILED DESCRIPTION

One difficulty in treating GBM is a lack of therapies that can overcome the inter- and the intra-tumoral heterogeneity and plasticity presented in GBM. Here, it has been discovered that HDAC-7 is highly expressed in glioblastoma compared to normal brain tissue. It has also been discovered that inhibiting HDAC-7 (Histone Deacetylase 7) has a significant effect on GBM growth, and inhibits GSC properties, transcriptomic expression, and invasion of cancer cells.

In addition, it has been discovered that inhibiting HDAC-7 or CLOCK (Circadian Locomotor Output Cycles Kaput), or downregulating ASF1A (Anti-Silencing Function 1A Histone Chaperone), SUV39H2 (Suppressor Of Variegation 3-9 Homolog 2), or WHSC1L1 (Wolf-Hisrchhorn Syndrome Candidate 1-Like 1), presented a phenotype change in an in-vitro GSC culture and decreased the survival of an in-vivo GBM human xenograft mouse model.

Thus, in one embodiment, provided herein are GBM inhibitors that are HDAC-7 inhibitors. In some embodiments, provided herein are HDAC-7 inhibitors selected from small molecules, siRNAs, pharmaceutically acceptable salts thereof, or combinations thereof. In some embodiments, the inhibitors include HDAC class Ila inhibitors, such as TMP269, or a pharmaceutically acceptable salt thereof. In some embodiments, the inhibitors include HDAC-7 targeting siRNAs or a pharmaceutically acceptable salt thereof. In some embodiments, the inhibitors include siRNAs that target one or more of the following transcripts for HDAC7: NM_001098416; NM_001308090; NM_015401; XM_011538478; XM_011538479; XM_011538480; XM_011538481; XM_011538482; XM_011538483; XM_017019455; XM_017019456; or XR_001748761. In some embodiments, the siRNA comprises one or more of GACAAGAGCAAGCGAAGUG (SEQ ID NO:1), GCAGAUACCCUCGGCUGAA (SEQ ID NO:2), GGUGAGGGCUUCAAUGUCA (SEQ ID NO:3), or UGGCUGCUCUUCUGGGUAA (SEQ ID NO:4).

In addition to the foregoing inhibitors, a small molecule inhibitor library was designed that shows significant specificity towards HDAC-7 compared to other Class IIa Histone Deacetylases. These small molecule are disclosed in Table 1. Thus, in some embodiments, provided herein is a compound selected from Table 1, or a pharmaceutically acceptable salt thereof.

TABLE 1

| Compound | Structure |
|---|---|
| 1 | 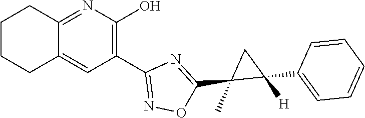 [SMILES: C[C@]1(c2nc(-c3cc4c(nc3O)CCCC4)no2)C[C@@H]1c1ccccc1] |
| 2 | 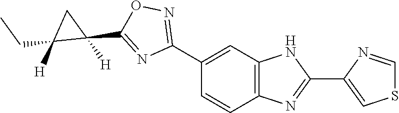 [SMILES: CC[C@@H]1C[C@H]1c1nc(-c2ccc3nc(-c4cscn4)[nH]c3c2)no1] |
| 3 | 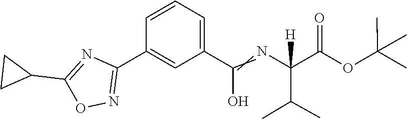 [SMILES: CC(C)[C@H](N=C(O)c1cccc(-c2noc(C3CC3)n2)c1)C(=O)OC(C)(C)C] |
| 4 | 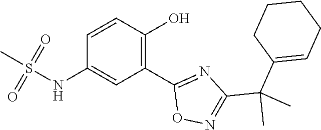 [SMILES: CC(C)(C1=CCCCC1)c1noc(-c2cc(NS(C)(=O)=O)ccc2O)n1] |
| 5 | 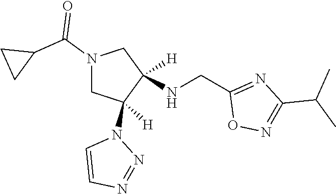 [SMILES: CC(C)c1noc(CN[C@@H]2CN(C(=O)C3CC3)C[C@@H]2n2ccnn2)n1] |
| 6 | 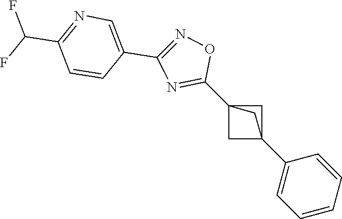 [SMILES: FC(F)c1ccc(-c2noc(C34CC(c5ccccc5)(C3)C4)n2)cn1] |
| 7 | 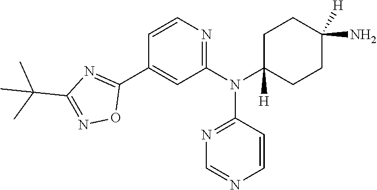 [SMILES: CC(C)(C)c1noc(-c2ccnc(N(c3ccncn3)[C@H]3CC[C@H](N)CC3)c2)n1] |
| 8 | 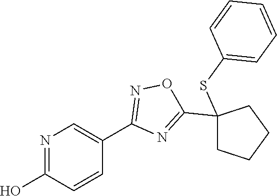 [SMILES: Oc1ccc(-c2noc(C3(Sc4ccccc4)CCCC3)n2)cn1] |
| 9 | 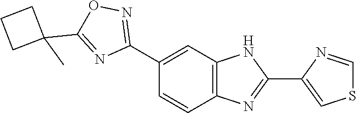 [SMILES: CC1(c2nc(-c3ccc4nc(-c5cscn5)[nH]c4c3)no2)CCC1] |
| 10 | 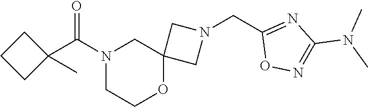 [SMILES: CN(C)c1noc(CN2CC3(C2)CN(C(=O)C2(C)CCC2)CCO3)n1] |
| 11 | 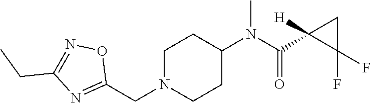 [SMILES: CCc1noc(CN2CCC(N(C)C(=O)[C@@H]3CC3(F)F)CC2)n1] |
| 12 | 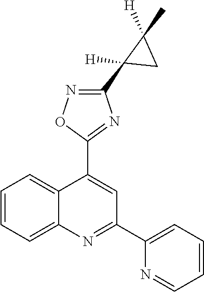 [SMILES: C[C@H]1C[C@H]1c1noc(-c2cc(-c3ccccn3)nc3ccccc23)n1] |
| 13 | 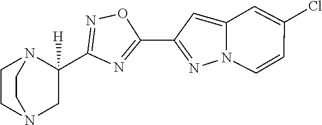 [SMILES: Clc1ccn2nc(-c3nc([C@H]4CN5CCN4CC5)no3)cc2c1] |
| 14 | 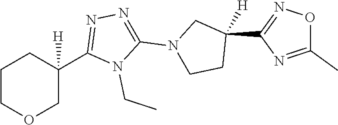 [SMILES: CCn1c([C@@H]2CCCOC2)nnc1N1CC[C@H](c2noc(C)n2)C1] |
| 15 | 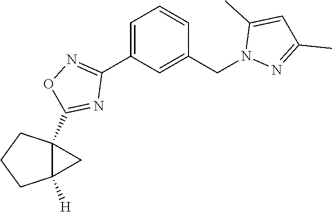 [SMILES: Cc1cc(C)n(Cc2cccc(-c3noc([C@@]45CCC[C@@H]4C5)n3)c2)n1] |
| 16 | 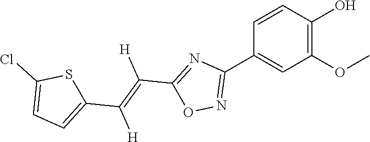 [SMILES: COc1cc(-c2noc(/C=C/c3ccc(Cl)s3)n2)ccc1O] |
| 17 | 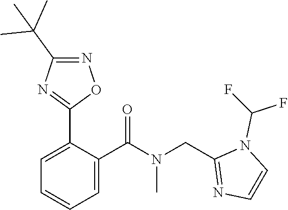 |
| 18 | 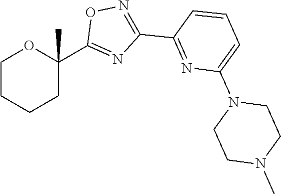 [SMILES: CN1CCN(c2cccc(-c3noc([C@]4(C)CCCCO4)n3)n2)CC1] |
| 19 | 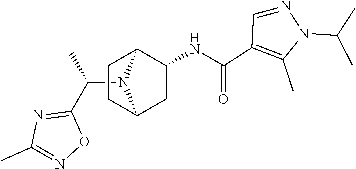 [SMILES: Cc1noc([C@H](C)N2[C@@H]3CC[C@H]2[C@H](NC(=O)c2cnn(C(C)C)c2C)C3)n1] |
| 20 | 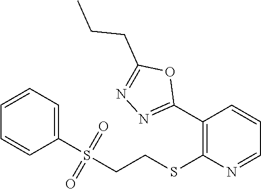 [SMILES: CCCc1nnc(-c2cccnc2SCCS(=O)(=O)c2ccccc2)o1] |
| 21 | 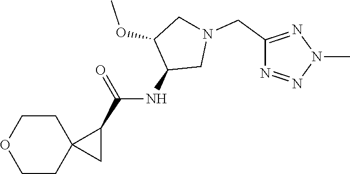 [SMILES: CO[C@@H]1CN(Cc2nnn(C)n2)C[C@H]1NC(=O)[C@H]1CC12CCOCC2] |
| 22 | 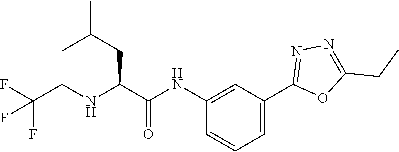 [SMILES: CCc1nnc(-c2cccc(NC(=O)[C@H](CC(C)C)NCC(F)(F)F)c2)o1] |
| 23 | 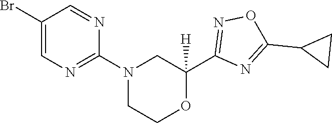 [SMILES: Brc1cnc(N2CCO[C@H](c3noc(C4CC4)n3)C2)nc1] |
| 24 | 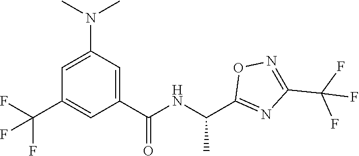 [SMILES: C[C@H](NC(=O)c1cc(N(C)C)cc(C(F)(F)F)c1)c1nc(C(F)(F)F)no1] |
| 25 | 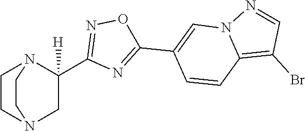 [SMILES: Brc1cnn2cc(-c3nc([C@H]4CN5CCN4CC5)no3)ccc12] |
| 26 | 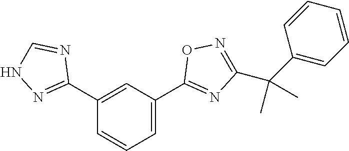 [SMILES: CC(C)(c1ccccc1)c1noc(-c2cccc(-c3nc[nH]n3)c2)n1] |
| 27 | 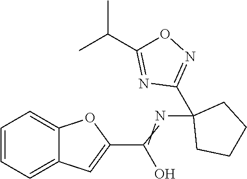 [SMILES: CC(C)c1nc(C2(N=C(O)c3cc4ccccc4o3)CCCC2)no1] |
| 28 | 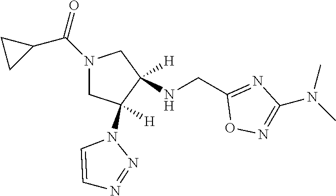 [SMILES: CN(C)c1noc(CN[C@@H]2CN(C(=O)C3CC3)C[C@@H]2n2ccnn2)n1] |
| 29 | 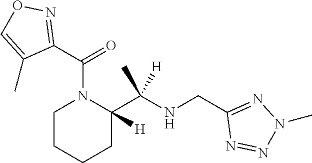 [SMILES: Cc1conc1C(=O)N1CCCC[C@H]1[C@@H](C)NCc1nnn(C)n1] |
| 30 | 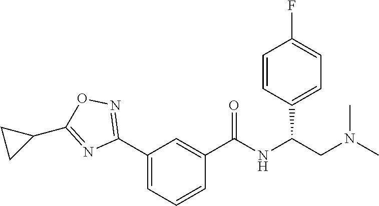 [SMILES: CN(C)C[C@H](NC(=O)c1cccc(-c2noc(C3CC3)n2)c1)c1ccc(F)cc1] |
| 31 | 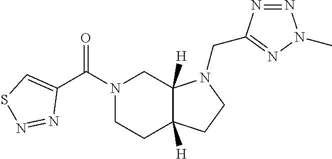 [SMILES: Cn1nnc(CN2CC[C@@H]3CCN(C(=O)c4csnn4)C[C@@H]32)n1] |
| 32 | 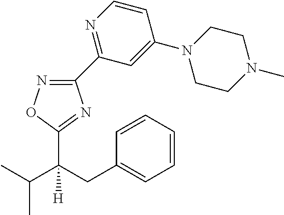 [SMILES: CC(C)[C@H](Cc1ccccc1)c1nc(-c2cc(N3CCN(C)CC3)ccn2)no1] |
| 33 | 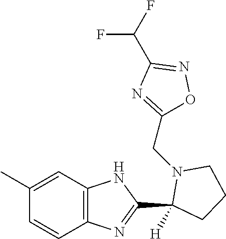 [SMILES: Cc1ccc2nc([C@@H]3CCCN3Cc3nc(C(F)F)no3)[nH]c2c1] |
| 34 | 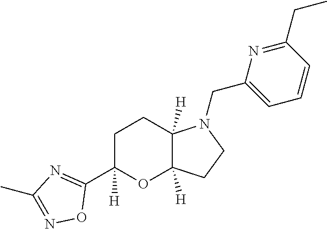 [SMILES: CCc1cccc(CN2CC[C@H]3O[C@@H](c4nc(C)no4)CC[C@H]32)n1] |
| 35 | 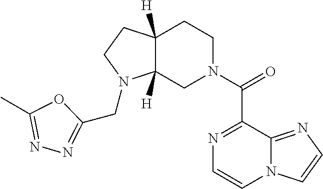 [SMILES: Cc1nnc(CN2CC[C@@H]3CCN(C(=O)c4nccn5ccnc45)C[C@@H]32)o1] |
| 36 | 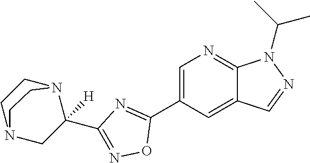 [SMILES: CC(C)n1ncc2cc(-c3nc([C@H]4CN5CCN4CC5)no3)cnc21] |
| 37 | 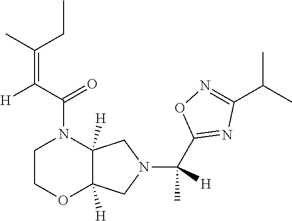 [SMILES: CC/C(C)=C\C(=O)N1CCO[C@@H]2CN([C@@H](C)c3nc(C(C)C)no3)C[C@@H]21] |
| 38 | 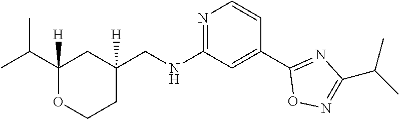 [SMILES: CC(C)c1noc(-c2ccnc(NC[C@H]3CCO[C@H](C(C)C)C3)c2)n1] |
| 39 | 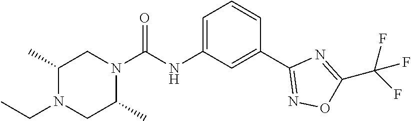 [SMILES: CCN1C[C@@H](C)N(C(=O)Nc2cccc(-c3noc(C(F)(F)F)n3)c2)C[C@H]1C] |
| 40 | 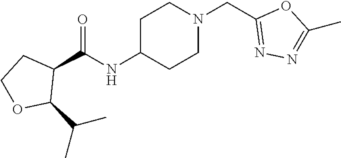 [SMILES: Cc1nnc(CN2CCC(NC(=O)[C@@H]3CCO[C@@H]3C(C)C)CC2)o1] |
| 41 | 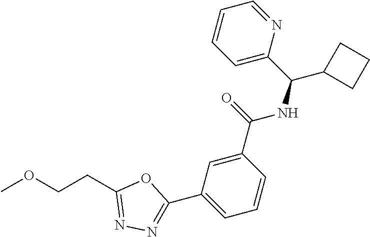 [SMILES: COCCc1nnc(-c2cccc(C(=O)N[C@@H](c3ccccn3)C3CCC3)c2)o1] |
| 42 | 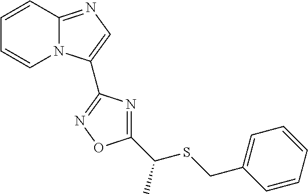 [SMILES: C[C@@H](SCc1ccccc1)c1nc(-c2cnc3ccccn23)no1] |
| 43 | 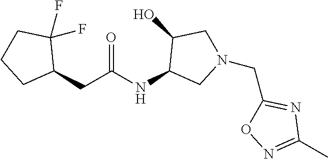 [SMILES: Cc1noc(CN2C[C@H](O)[C@H](NC(=O)C[C@H]3CCCC3(F)F)C2)n1] |
| 44 | 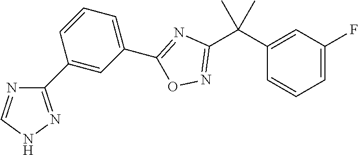 [SMILES: CC(C)(c1cccc(F)c1)c1noc(-c2cccc(-c3nc[nH]n3)c2)n1] |
| 45 | 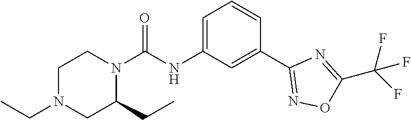 [SMILES: CC[C@H]1CN(CC)CCN1C(=O)Nc1cccc(-c2noc(C(F)(F)F)n2)c1] |
| 46 | 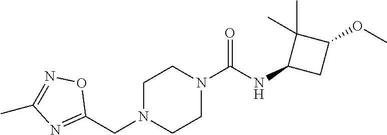 |
| 47 | 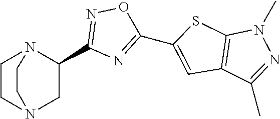 [SMILES: Cc1nn(C)c2sc(-c3nc([C@H]4CN5CCN4CC5)no3)cc12] |
| 48 | 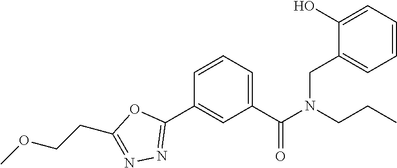 [SMILES: CCCN(Cc1ccccc1O)C(=O)c1cccc(-c2nnc(CCOC)o2)c1] |
| 49 | 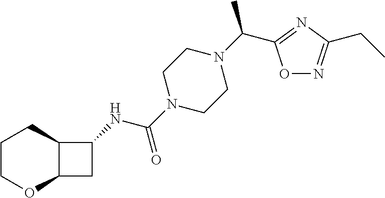 [SMILES: CCc1noc([C@H](C)N2CCN(C(=O)N[C@@H]3C[C@H]4OCCC[C@@H]34)CC2)n1] |
| 50 | 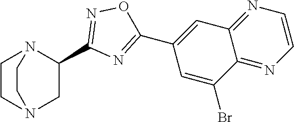 [SMILES: Brc1cc(-c2nc([C@H]3CN4CCN3CC4)no2)cc2nccnc12] |
| 51 | 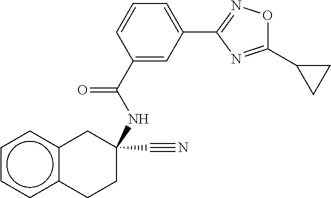 |
| 52 | 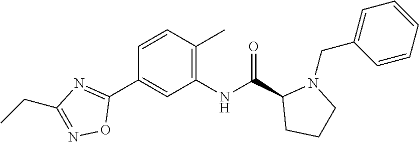 [SMILES: CCc1noc(-c2ccc(C)c(NC(=O)[C@@H]3CCCN3Cc3ccccc3)c2)n1] |
| 53 | 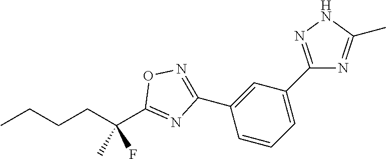 [SMILES: CCCC[C@](C)(F)c1nc(-c2cccc(-c3n[nH]c(C)n3)c2)no1] |
| 54 | 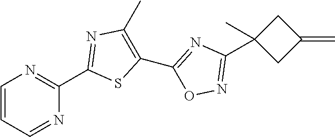 [SMILES: C=C1CC(C)(c2noc(-c3sc(-c4ncccn4)nc3C)n2)C1] |
| 55 | 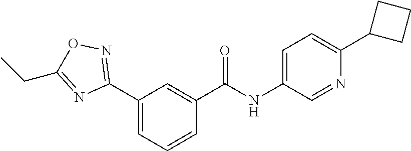 [SMILES: CCc1nc(-c2cccc(C(=O)Nc3ccc(C4CCC4)nc3)c2)no1] |
| 56 | 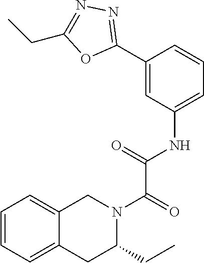 [SMILES: CCc1nnc(-c2cccc(NC(=O)C(=O)N3Cc4ccccc4C[C@H]3CC)c2)o1] |
| 57 | 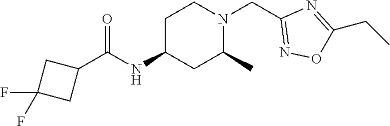 [SMILES: CCc1nc(CN2CC[C@H](NC(=O)C3CC(F)(F)C3)C[C@@H]2C)no1] |
| 58 | 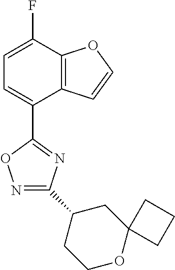 [SMILES: Fc1ccc(-c2nc([C@H]3CCOC4(CCC4)C3)no2)c2ccoc12] |
| 59 | 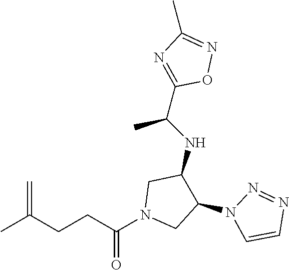 [SMILES: C=C(C)CCC(=O)N1C[C@@H](N[C@@H](C)c2nc(C)no2)[C@@H](n2ccnn2)C1] |
| 60 | 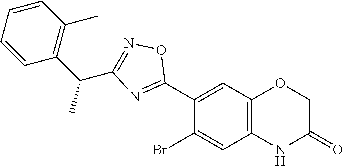 [SMILES: Cc1ccccc1[C@@H](C)c1noc(-c2cc3c(cc2Br)NC(=O)CO3)n1] |
| 61 | 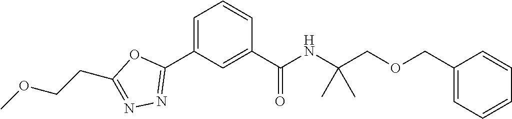 [SMILES: COCCc1nnc(-c2cccc(C(=O)NC(C)(C)COCc3ccccc3)c2)o1] |
| 62 | 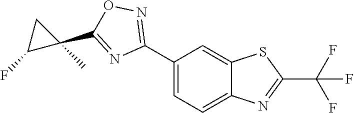 [SMILES: C[C@@]1(c2nc(-c3ccc4nc(C(F)(F)F)sc4c3)no2)C[C@H]1F] |
| 63 | 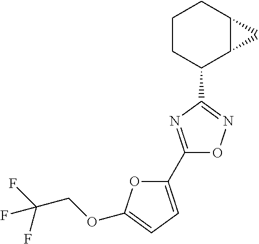 [SMILES: FC(F)(F)COc1ccc(-c2nc([C@@H]3CCC[C@@H]4C[C@@H]43)no2)o1] |
| 64 | 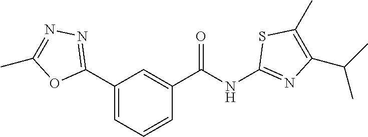 [SMILES: Cc1nnc(-c2cccc(C(=O)Nc3nc(C(C)C)c(C)s3)c2)o1] |
| 65 | 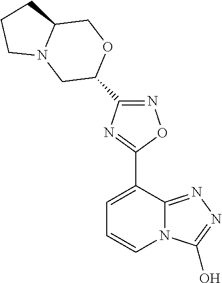 [SMILES: Oc1nnc2c(-c3nc([C@@H]4CN5CCC[C@H]5CO4)no3)cccn12] |
| 66 | 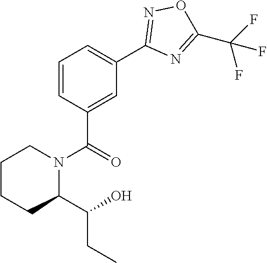 [SMILES: CC[C@@H](O)[C@H]1CCCCN1C(=O)c1cccc(-c2noc(C(F)(F)F)n2)c1] |
| 67 | 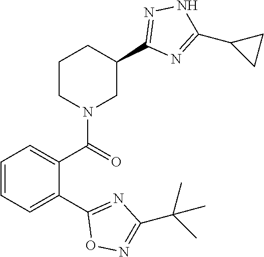 [SMILES: CC(C)(C)c1noc(-c2ccccc2C(=O)N2CCC[C@@H](c3n[nH]c(C4CC4)n3)C2)n1] |
| 68 | 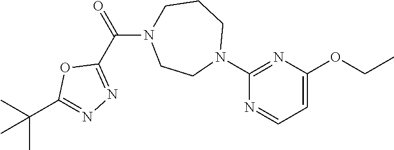 [SMILES: CCOc1ccnc(N2CCCN(C(=O)c3nnc(C(C)(C)C)o3)CC2)n1] |
| 69 | 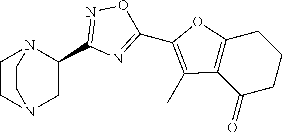 [SMILES: Cc1c(-c2nc([C@H]3CN4CCN3CC4)no2)oc2c1C(=O)CCC2] |
| 70 | 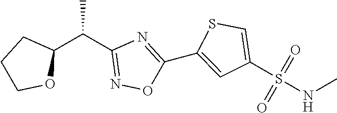 [SMILES: CNS(=O)(=O)c1csc(-c2nc([C@@H](C)[C@@H]3CCCO3)no2)c1] |
| 71 | 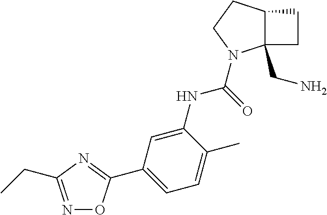 [SMILES: CCc1noc(-c2ccc(C)c(NC(=O)N3CC[C@@H]4CC[C@@]43CN)c2)n1] |
| 72 | 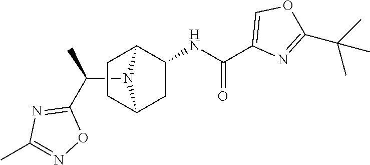 [SMILES: Cc1noc([C@@H](C)N2[C@@H]3CC[C@H]2[C@H](NC(=O)c2coc(C(C)(C)C)n2)C3)n1] |
| 73 | 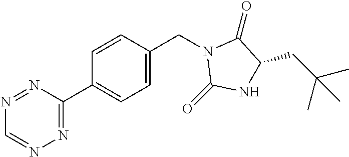 [SMILES: CC(C)(C)C[C@@H]1NC(=O)N(Cc2ccc(-c3nncnn3)cc2)C1=O] |
| 74 | 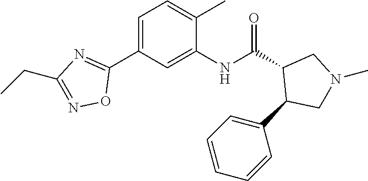 [SMILES: CCc1noc(-c2ccc(C)c(NC(=O)[C@@H]3CN(C)C[C@H]3c3ccccc3)c2)n1] |
| 75 | 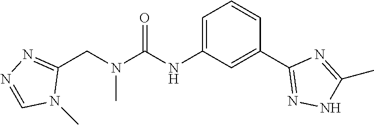 [SMILES: Cc1nc(-c2cccc(NC(=O)N(C)Cc3nncn3C)c2)n[nH]1] |
| 76 | 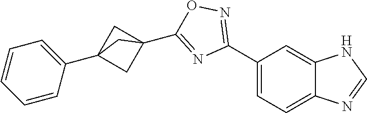 [SMILES: c1ccc(C23CC(c4nc(-c5ccc6nc[nH]c6c5)no4)(C2)C3)cc1] |
| 77 | 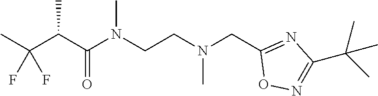 [SMILES: C[C@H](C(=O)N(C)CCN(C)Cc1nc(C(C)(C)C)no1)C(C)(F)F] |
| 78 | 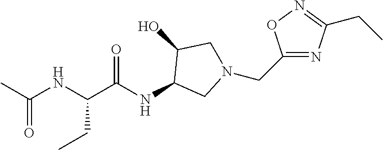 [SMILES: CCc1noc(CN2C[C@H](O)[C@H](NC(=O)[C@H](CC)NC(C)=O)C2)n1] |
| 79 | 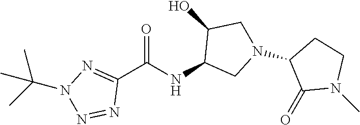 [SMILES: CN1CC[C@@H](N2C[C@H](O)[C@H](NC(=O)c3nnn(C(C)(C)C)n3)C2)C1=O] |
| 80 | 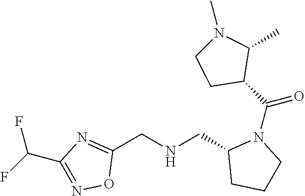 [SMILES: C[C@@H]1[C@H](C(=O)N2CCC[C@@H]2CNCc2nc(C(F)F)no2)CCN1C] |
| 81 | 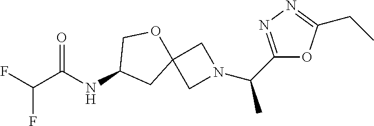 [SMILES: CCc1nnc([C@@H](C)N2CC3(C[C@@H](NC(=O)C(F)F)CO3)C2)o1] |
| 82 | 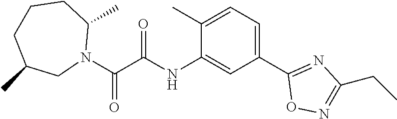 [SMILES: CCc1noc(-c2ccc(C)c(NC(=O)C(=O)N3C[C@@H](C)CCC[C@@H]3C)c2)n1] |
| 83 | 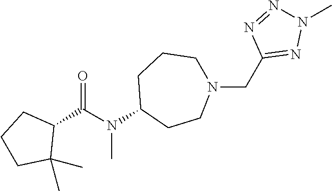 [SMILES: CN(C(=O)[C@H]1CCCC1(C)C)[C@@H]1CCCN(Cc2nnn(C)n2)CC1] |
| 84 | 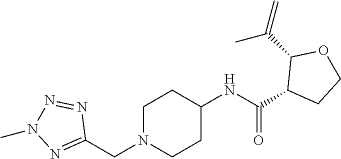 [SMILES: C=C(C)[C@@H]1OCC[C@@H]1C(=O)NC1CCN(Cc2nnn(C)n2)CC1] |
| 85 | 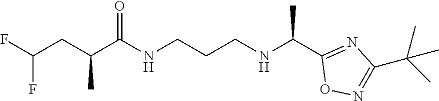 [SMILES: C[C@H](NCCCNC(=O)[C@@H](C)CC(F)F)c1nc(C(C)(C)C)no1] |
| 86 | 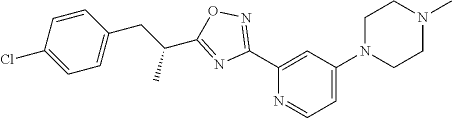 [SMILES: C[C@H](Cc1ccc(Cl)cc1)c1nc(-c2cc(N3CCN(C)CC3)ccn2)no1] |
| 87 | 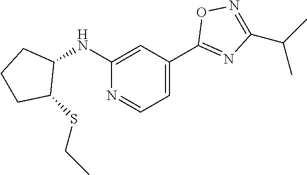 [SMILES: CCS[C@@H]1CCC[C@@H]1Nc1cc(-c2nc(C(C)C)no2)ccn1] |
| 88 | 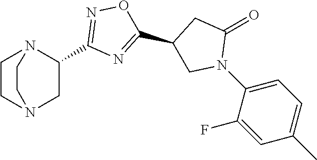 [SMILES: Cc1ccc(N2C[C@@H](c3nc([C@@H]4CN5CCN4CC5)no3)CC2=O)c(F)c1] |
| 89 | 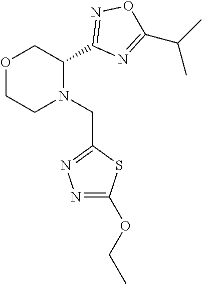 [SMILES: CCOc1nnc(CN2CCOC[C@@H]2c2noc(C(C)C)n2)s1] |
| 90 | 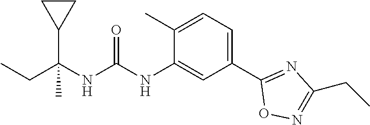 [SMILES: CCc1noc(-c2ccc(C)c(NC(=O)N[C@@](C)(CC)C3CC3)c2)n1] |
| 91 | 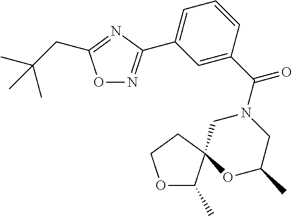 [SMILES: C[C@@H]1CN(C(=O)c2cccc(-c3noc(CC(C)(C)C)n3)c2)C[C@]2(CCO[C@H]2C)O1] |
| 92 | 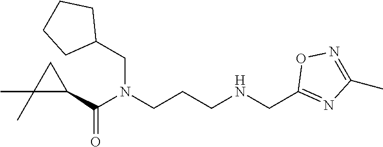 |
| 93 | 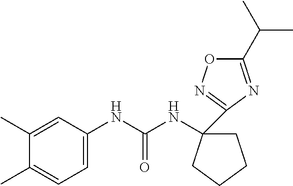 [SMILES: Cc1ccc(NC(=O)NC2(c3noc(C(C)C)n3)CCCC2)cc1C] |
| 94 | 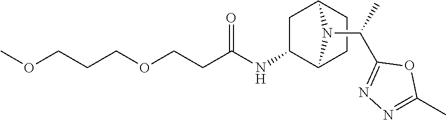 |
| 95 | 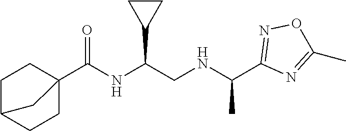 [SMILES: Cc1nc([C@@H](C)NC[C@@H](NC(=O)C23CCC(CC2)C3)C2CC2)no1] |
| 96 | 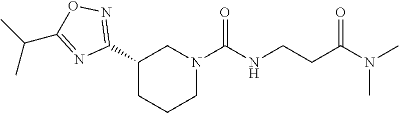 [SMILES: CC(C)c1nc([C@H]2CCCN(C(=O)NCCC(=O)N(C)C)C2)no1] |
| 97 | 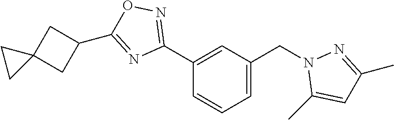 [SMILES: Cc1cc(C)n(Cc2cccc(-c3noc(C4CC5(CC5)C4)n3)c2)n1] |
| 98 | 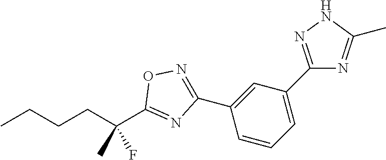 [SMILES: CCCC[C@@](C)(F)c1nc(-c2cccc(-c3n[nH]c(C)n3)c2)no1] |
| 99 | 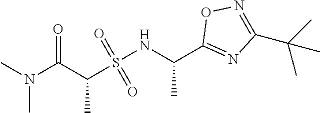 [SMILES: C[C@H](NS(=O)(=O)[C@H](C)C(=O)N(C)C)c1nc(C(C)(C)C)no1] |
| 100 | 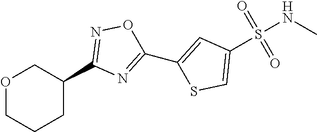 [SMILES: CNS(=O)(=O)c1csc(-c2nc([C@H]3CCCOC3)no2)c1] |

HDAC-7 has been crystalized and similar to other members of class I, II, and IV, HDAC-7 binds to the substrates through a zinc mediated charge relay system. Class IIa HDAC has another specific zinc-binding motif adjacent to the active site, which directs the recognition and protein-protein interaction, and is most likely the site for docking of cofactors and regulatory proteins, and that would provide a site for modulation of activity. A remarkable structural diversity in HDAC-7 is found in loop regions around the active site entrance that unconventionally mediate monodentate, rather than other HDACs' bidentate coordination of substrates, and thus, are likely to be mediating substrate binding and specificity.

To design novel small molecule inhibitors, such as those in Table 1, with increased specificity against HDAC-7 compared to other HDAC Class IIa members, a structure-based virtual screening of the structurally related compounds of HDAC IIa inhibitors was conducted. The screen was performed against a 1.5 billion digital compound library comparing binding to HDAC-7 with HDAC-4 and HDAC-5. The hits were also compared to the inhibitory binding of the commercially available pan-Class IIa HDAC inhibitor TMP269. By superposing the crystal structures together, we compared the chemical binding of different binding groups to TMP269 to evaluate whether any potential binding groups will increase the affinity towards HDAC-7 and decrease the affinity towards HDAC-4 and HDAC-5. The latter approach was applied in a sequential pipeline by looking up the structural differences of the inhibitor binding upon the input HDAC structures' superposition. Then the Schrodinger computational ligand designer software was used to integrate predictive physics-based methods with machine learning techniques in combinatorial chemical binding, docking, affinity calculations, and modeling the potential compounds HDAC-7 versus HDAC-4 and HDAC-5. Thus, instead of manually predicting the sites that tolerate the extension of binding groups in TMP269, the software provides a reliable prediction. By trying many binding groups in these predicted sites, compounds with a higher affinity towards HDAC-7 were successfully docked. Finally, to calculate the physical interaction and the energy perturbation for the affinity calculations in these compounds towards HDAC-7, the more complicated library for the Schrodinger software that integrates more sophisticated considerations in the binding like the dynamic simulation and interactions with water molecules, neighboring ions, and needed cofactors, was used. This approach yielded more applicable “druggable” compounds that could show better pharmacokinetics and pharmacodynamics when used biologically.

In some embodiment, the compounds described herein, e.g., the HDAC-7 inhibitors or their salts, exist as tautomers.

Compounds described herein also include isotopically-labeled compounds wherein one or more atoms is replaced by an atom having the same atomic number, but an atomic mass or mass number different from the atomic mass or mass number predominantly found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In some embodiments, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In some embodiments, reactive functional groups, such as hydroxyl, amino, imino, thio, or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In some embodiments, protective groups are removed by acid, base, reducing conditions (for example, by hydrogenolysis), or oxidative conditions. Groups such as trityl, dimethoxytrityl, acetal, and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

Thus, in some embodiments, provided herein is compound 1 of Table 1, or a pharmaceutically acceptable salt thereof. In some embodiments, compound 1 or its pharmaceutically acceptable salt is useful in treating brain cancer, including, e.g., GBM.

In some embodiments, provided herein is compound 2 of Table 1, or a pharmaceutically acceptable salt thereof. In some embodiments, compound 2 or its pharmaceutically acceptable salt is useful in treating brain cancer, including, e.g., GBM.

In some embodiments, provided herein is compound 3 of Table 1, or a pharmaceutically acceptable salt thereof. In some embodiments, compound 3 or its pharmaceutically acceptable salt is useful in treating brain cancer, including, e.g., GBM.

In some embodiments, provided herein is compound 4 of Table 1, or a pharmaceutically acceptable salt thereof. In some embodiments, compound 4 or its pharmaceutically acceptable salt is useful in treating brain cancer, including, e.g., GBM.

In some embodiments, provided herein is compound 5 of Table 1, or a pharmaceutically acceptable salt thereof. In some embodiments, compound 5 or its pharmaceutically acceptable salt is useful in treating brain cancer, including, e.g., GBM.

In some embodiments, provided herein is compound 6 of Table 1, or a pharmaceutically acceptable salt thereof. In some embodiments, compound 6 or its pharmaceutically acceptable salt is useful in treating brain cancer, including, e.g., GBM.

In some embodiments, provided herein is compound 7 of Table 1, or a pharmaceutically acceptable salt thereof. In some embodiments, compound 7 or its pharmaceutically acceptable salt is useful in treating brain cancer, including, e.g., GBM.

In some embodiments, provided herein is compound 8 of Table 1, or a pharmaceutically acceptable salt thereof. In some embodiments, compound 8 or its pharmaceutically acceptable salt is useful in treating brain cancer, including, e.g., GBM.

In some embodiments, provided herein is compound 9 of Table 1, or a pharmaceutically acceptable salt thereof. In some embodiments, compound 9 or its pharmaceutically acceptable salt is useful in treating brain cancer, including, e.g., GBM.

In some embodiments, provided herein is compound 10 of Table 1, or a pharmaceutically acceptable salt thereof. In some embodiments, compound 10 or its pharmaceutically acceptable salt is useful in treating brain cancer, including, e.g., GBM.

In some embodiments, provided herein is compound 11 of Table 1, or a pharmaceutically acceptable salt thereof. In some embodiments, compound 11 or its pharmaceutically acceptable salt is useful in treating brain cancer, including, e.g., GBM.

In some embodiments, provided herein is compound 12 of Table 1, or a pharmaceutically acceptable salt thereof. In some embodiments, compound 12 or its pharmaceutically acceptable salt is useful in treating brain cancer, including, e.g., GBM.

In some embodiments, provided herein is compound 13 of Table 1, or a pharmaceutically acceptable salt thereof. In some embodiments, compound 13 or its pharmaceutically acceptable salt is useful in treating brain cancer, including, e.g., GBM.

In some embodiments, provided herein is compound 14 of Table 1, or a pharmaceutically acceptable salt thereof. In some embodiments, compound 14 or its pharmaceutically acceptable salt is useful in treating brain cancer, including, e.g., GBM.

In some embodiments, provided herein is compound 15 of Table 1, or a pharmaceutically acceptable salt thereof. In some embodiments, compound 15 or its pharmaceutically acceptable salt is useful in treating brain cancer, including, e.g., GBM.

In some embodiments, provided herein is compound 16 of Table 1, or a pharmaceutically acceptable salt thereof. In some embodiments, compound 16 or its pharmaceutically acceptable salt is useful in treating brain cancer, including, e.g., GBM.

In some embodiments, provided herein is compound 17 of Table 1, or a pharmaceutically acceptable salt thereof. In some embodiments, compound 17 or its pharmaceutically acceptable salt is useful in treating brain cancer, including, e.g., GBM.

In some embodiments, provided herein is compound 18 of Table 1, or a pharmaceutically acceptable salt thereof. In some embodiments, compound 18 or its pharmaceutically acceptable salt is useful in treating brain cancer, including, e.g., GBM.

In some embodiments, provided herein is compound 19 of Table 1, or a pharmaceutically acceptable salt thereof. In some embodiments, compound 19 or its pharmaceutically acceptable salt is useful in treating brain cancer, including, e.g., GBM.

In some embodiments, provided herein is compound 20 of Table 1, or a pharmaceutically acceptable salt thereof. In some embodiments, compound 20 or its pharmaceutically acceptable salt is useful in treating brain cancer, including, e.g., GBM.

In some embodiments, provided herein is compound 21 of Table 1, or a pharmaceutically acceptable salt thereof. In some embodiments, compound 21 or its pharmaceutically acceptable salt is useful in treating brain cancer, including, e.g., GBM.

In some embodiments, provided herein is compound 22 of Table 1, or a pharmaceutically acceptable salt thereof. In some embodiments, compound 22 or its pharmaceutically acceptable salt is useful in treating brain cancer, including, e.g., GBM.

In some embodiments, provided herein is compound 23 of Table 1, or a pharmaceutically acceptable salt thereof. In some embodiments, compound 23 or its pharmaceutically acceptable salt is useful in treating brain cancer, including, e.g., GBM.

In some embodiments, provided herein is compound 24 of Table 1, or a pharmaceutically acceptable salt thereof. In some embodiments, compound 24 or its pharmaceutically acceptable salt is useful in treating brain cancer, including, e.g., GBM.

In some embodiments, provided herein is compound 25 of Table 1, or a pharmaceutically acceptable salt thereof. In some embodiments, compound 25 or its pharmaceutically acceptable salt is useful in treating brain cancer, including, e.g., GBM.

In some embodiments, provided herein is compound 26 of Table 1, or a pharmaceutically acceptable salt thereof. In some embodiments, compound 26 or its pharmaceutically acceptable salt is useful in treating brain cancer, including, e.g., GBM.

In some embodiments, provided herein is compound 27 of Table 1, or a pharmaceutically acceptable salt thereof. In some embodiments, compound 27 or its pharmaceutically acceptable salt is useful in treating brain cancer, including, e.g., GBM.

In some embodiments, provided herein is compound 28 of Table 1, or a pharmaceutically acceptable salt thereof. In some embodiments, compound 28 or its pharmaceutically acceptable salt is useful in treating brain cancer, including, e.g., GBM.

In some embodiments, provided herein is compound 29 of Table 1, or a pharmaceutically acceptable salt thereof. In some embodiments, compound 29 or its pharmaceutically acceptable salt is useful in treating brain cancer, including, e.g., GBM.

In some embodiments, provided herein is compound 30 of Table 1, or a pharmaceutically acceptable salt thereof. In some embodiments, compound 30 or its pharmaceutically acceptable salt is useful in treating brain cancer, including, e.g., GBM.

In some embodiments, provided herein is compound 31 of Table 1, or a pharmaceutically acceptable salt thereof. In some embodiments, compound 31 or its pharmaceutically acceptable salt is useful in treating brain cancer, including, e.g., GBM.

In some embodiments, provided herein is compound 32 of Table 1, or a pharmaceutically acceptable salt thereof. In some embodiments, compound 32 or its pharmaceutically acceptable salt is useful in treating brain cancer, including, e.g., GBM.

In some embodiments, provided herein is compound 33 of Table 1, or a pharmaceutically acceptable salt thereof. In some embodiments, compound 33 or its pharmaceutically acceptable salt is useful in treating brain cancer, including, e.g., GBM.

In some embodiments, provided herein is compound 34 of Table 1, or a pharmaceutically acceptable salt thereof. In some embodiments, compound 34 or its pharmaceutically acceptable salt is useful in treating brain cancer, including, e.g., GBM.

In some embodiments, provided herein is compound 35 of Table 1, or a pharmaceutically acceptable salt thereof. In some embodiments, compound 35 or its pharmaceutically acceptable salt is useful in treating brain cancer, including, e.g., GBM.

In some embodiments, provided herein is compound 36 of Table 1, or a pharmaceutically acceptable salt thereof. In some embodiments, compound 36 or its pharmaceutically acceptable salt is useful in treating brain cancer, including, e.g., GBM.

In some embodiments, provided herein is compound 37 of Table 1, or a pharmaceutically acceptable salt thereof. In some embodiments, compound 37 or its pharmaceutically acceptable salt is useful in treating brain cancer, including, e.g., GBM.

In some embodiments, provided herein is compound 38 of Table 1, or a pharmaceutically acceptable salt thereof. In some embodiments, compound 38 or its pharmaceutically acceptable salt is useful in treating brain cancer, including, e.g., GBM.

In some embodiments, provided herein is compound 39 of Table 1, or a pharmaceutically acceptable salt thereof. In some embodiments, compound 39 or its pharmaceutically acceptable salt is useful in treating brain cancer, including, e.g., GBM.

In some embodiments, provided herein is compound 40 of Table 1, or a pharmaceutically acceptable salt thereof. In some embodiments, compound 40 or its pharmaceutically acceptable salt is useful in treating brain cancer, including, e.g., GBM.

In some embodiments, provided herein is compound 41 of Table 1, or a pharmaceutically acceptable salt thereof. In some embodiments, compound 41 or its pharmaceutically acceptable salt is useful in treating brain cancer, including, e.g., GBM.

In some embodiments, provided herein is compound 42 of Table 1, or a pharmaceutically acceptable salt thereof. In some embodiments, compound 42 or its pharmaceutically acceptable salt is useful in treating brain cancer, including, e.g., GBM.

In some embodiments, provided herein is compound 43 of Table 1, or a pharmaceutically acceptable salt thereof. In some embodiments, compound 43 or its pharmaceutically acceptable salt is useful in treating brain cancer, including, e.g., GBM.

In some embodiments, provided herein is compound 44 of Table 1, or a pharmaceutically acceptable salt thereof. In some embodiments, compound 44 or its pharmaceutically acceptable salt is useful in treating brain cancer, including, e.g., GBM.

In some embodiments, provided herein is compound 45 of Table 1, or a pharmaceutically acceptable salt thereof. In some embodiments, compound 45 or its pharmaceutically acceptable salt is useful in treating brain cancer, including, e.g., GBM.

In some embodiments, provided herein is compound 46 of Table 1, or a pharmaceutically acceptable salt thereof. In some embodiments, compound 46 or its pharmaceutically acceptable salt is useful in treating brain cancer, including, e.g., GBM.

In some embodiments, provided herein is compound 47 of Table 1, or a pharmaceutically acceptable salt thereof. In some embodiments, compound 47 or its pharmaceutically acceptable salt is useful in treating brain cancer, including, e.g., GBM.

In some embodiments, provided herein is compound 48 of Table 1, or a pharmaceutically acceptable salt thereof. In some embodiments, compound 48 or its pharmaceutically acceptable salt is useful in treating brain cancer, including, e.g., GBM.

In some embodiments, provided herein is compound 49 of Table 1, or a pharmaceutically acceptable salt thereof. In some embodiments, compound 49 or its pharmaceutically acceptable salt is useful in treating brain cancer, including, e.g., GBM.

In some embodiments, provided herein is compound 50 of Table 1, or a pharmaceutically acceptable salt thereof. In some embodiments, compound 50 or its pharmaceutically acceptable salt is useful in treating brain cancer, including, e.g., GBM.

In some embodiments, provided herein is compound 51 of Table 1, or a pharmaceutically acceptable salt thereof. In some embodiments, compound 51 or its pharmaceutically acceptable salt is useful in treating brain cancer, including, e.g., GBM.

In some embodiments, provided herein is compound 52 of Table 1, or a pharmaceutically acceptable salt thereof. In some embodiments, compound 52 or its pharmaceutically acceptable salt is useful in treating brain cancer, including, e.g., GBM.

In some embodiments, provided herein is compound 53 of Table 1, or a pharmaceutically acceptable salt thereof. In some embodiments, compound 53 or its pharmaceutically acceptable salt is useful in treating brain cancer, including, e.g., GBM.

In some embodiments, provided herein is compound 54 of Table 1, or a pharmaceutically acceptable salt thereof. In some embodiments, compound 54 or its pharmaceutically acceptable salt is useful in treating brain cancer, including, e.g., GBM.

In some embodiments, provided herein is compound 55 of Table 1, or a pharmaceutically acceptable salt thereof. In some embodiments, compound 55 or its pharmaceutically acceptable salt is useful in treating brain cancer, including, e.g., GBM.

In some embodiments, provided herein is compound 56 of Table 1, or a pharmaceutically acceptable salt thereof. In some embodiments, compound 56 or its pharmaceutically acceptable salt is useful in treating brain cancer, including, e.g., GBM.

In some embodiments, provided herein is compound 57 of Table 1, or a pharmaceutically acceptable salt thereof. In some embodiments, compound 57 or its pharmaceutically acceptable salt is useful in treating brain cancer, including, e.g., GBM.

In some embodiments, provided herein is compound 58 of Table 1, or a pharmaceutically acceptable salt thereof. In some embodiments, compound 58 or its pharmaceutically acceptable salt is useful in treating brain cancer, including, e.g., GBM.

In some embodiments, provided herein is compound 59 of Table 1, or a pharmaceutically acceptable salt thereof. In some embodiments, compound 59 or its pharmaceutically acceptable salt is useful in treating brain cancer, including, e.g., GBM.

In some embodiments, provided herein is compound 60 of Table 1, or a pharmaceutically acceptable salt thereof. In some embodiments, compound 60 or its pharmaceutically acceptable salt is useful in treating brain cancer, including, e.g., GBM.

In some embodiments, provided herein is compound 61 of Table 1, or a pharmaceutically acceptable salt thereof. In some embodiments, compound 61 or its pharmaceutically acceptable salt is useful in treating brain cancer, including, e.g., GBM.

In some embodiments, provided herein is compound 62 of Table 1, or a pharmaceutically acceptable salt thereof. In some embodiments, compound 62 or its pharmaceutically acceptable salt is useful in treating brain cancer, including, e.g., GBM.

In some embodiments, provided herein is compound 63 of Table 1, or a pharmaceutically acceptable salt thereof. In some embodiments, compound 63 or its pharmaceutically acceptable salt is useful in treating brain cancer, including, e.g., GBM.

In some embodiments, provided herein is compound 64 of Table 1, or a pharmaceutically acceptable salt thereof. In some embodiments, compound 64 or its pharmaceutically acceptable salt is useful in treating brain cancer, including, e.g., GBM.

In some embodiments, provided herein is compound 65 of Table 1, or a pharmaceutically acceptable salt thereof. In some embodiments, compound 65 or its pharmaceutically acceptable salt is useful in treating brain cancer, including, e.g., GBM.

In some embodiments, provided herein is compound 66 of Table 1, or a pharmaceutically acceptable salt thereof. In some embodiments, compound 66 or its pharmaceutically acceptable salt is useful in treating brain cancer, including, e.g., GBM.

In some embodiments, provided herein is compound 67 of Table 1, or a pharmaceutically acceptable salt thereof. In some embodiments, compound 67 or its pharmaceutically acceptable salt is useful in treating brain cancer, including, e.g., GBM.

In some embodiments, provided herein is compound 68 of Table 1, or a pharmaceutically acceptable salt thereof. In some embodiments, compound 68 or its pharmaceutically acceptable salt is useful in treating brain cancer, including, e.g., GBM.

In some embodiments, provided herein is compound 69 of Table 1, or a pharmaceutically acceptable salt thereof. In some embodiments, compound 69 or its pharmaceutically acceptable salt is useful in treating brain cancer, including, e.g., GBM.

In some embodiments, provided herein is compound 70 of Table 1, or a pharmaceutically acceptable salt thereof. In some embodiments, compound 70 or its pharmaceutically acceptable salt is useful in treating brain cancer, including, e.g., GBM.

In some embodiments, provided herein is compound 71 of Table 1, or a pharmaceutically acceptable salt thereof. In some embodiments, compound 71 or its pharmaceutically acceptable salt is useful in treating brain cancer, including, e.g., GBM.

In some embodiments, provided herein is compound 72 of Table 1, or a pharmaceutically acceptable salt thereof. In some embodiments, compound 72 or its pharmaceutically acceptable salt is useful in treating brain cancer, including, e.g., GBM.

In some embodiments, provided herein is compound 73 of Table 1, or a pharmaceutically acceptable salt thereof. In some embodiments, compound 73 or its pharmaceutically acceptable salt is useful in treating brain cancer, including, e.g., GBM.

In some embodiments, provided herein is compound 74 of Table 1, or a pharmaceutically acceptable salt thereof. In some embodiments, compound 74 or its pharmaceutically acceptable salt is useful in treating brain cancer, including, e.g., GBM.

In some embodiments, provided herein is compound 75 of Table 1, or a pharmaceutically acceptable salt thereof. In some embodiments, compound 75 or its pharmaceutically acceptable salt is useful in treating brain cancer, including, e.g., GBM.

In some embodiments, provided herein is compound 76 of Table 1, or a pharmaceutically acceptable salt thereof. In some embodiments, compound 76 or its pharmaceutically acceptable salt is useful in treating brain cancer, including, e.g., GBM.

In some embodiments, provided herein is compound 77 of Table 1, or a pharmaceutically acceptable salt thereof. In some embodiments, compound 77 or its pharmaceutically acceptable salt is useful in treating brain cancer, including, e.g., GBM.

In some embodiments, provided herein is compound 78 of Table 1, or a pharmaceutically acceptable salt thereof. In some embodiments, compound 78 or its pharmaceutically acceptable salt is useful in treating brain cancer, including, e.g., GBM.

In some embodiments, provided herein is compound 79 of Table 1, or a pharmaceutically acceptable salt thereof. In some embodiments, compound 79 or its pharmaceutically acceptable salt is useful in treating brain cancer, including, e.g., GBM.

In some embodiments, provided herein is compound 80 of Table 1, or a pharmaceutically acceptable salt thereof. In some embodiments, compound 80 or its pharmaceutically acceptable salt is useful in treating brain cancer, including, e.g., GBM.

In some embodiments, provided herein is compound 81 of Table 1, or a pharmaceutically acceptable salt thereof. In some embodiments, compound 81 or its pharmaceutically acceptable salt is useful in treating brain cancer, including, e.g., GBM.

In some embodiments, provided herein is compound 82 of Table 1, or a pharmaceutically acceptable salt thereof. In some embodiments, compound 82 or its pharmaceutically acceptable salt is useful in treating brain cancer, including, e.g., GBM.

In some embodiments, provided herein is compound 83 of Table 1, or a pharmaceutically acceptable salt thereof. In some embodiments, compound 83 or its pharmaceutically acceptable salt is useful in treating brain cancer, including, e.g., GBM.

In some embodiments, provided herein is compound 84 of Table 1, or a pharmaceutically acceptable salt thereof. In some embodiments, compound 84 or its pharmaceutically acceptable salt is useful in treating brain cancer, including, e.g., GBM.

In some embodiments, provided herein is compound 85 of Table 1, or a pharmaceutically acceptable salt thereof. In some embodiments, compound 85 or its pharmaceutically acceptable salt is useful in treating brain cancer, including, e.g., GBM.

In some embodiments, provided herein is compound 86 of Table 1, or a pharmaceutically acceptable salt thereof. In some embodiments, compound 86 or its pharmaceutically acceptable salt is useful in treating brain cancer, including, e.g., GBM.

In some embodiments, provided herein is compound 87 of Table 1, or a pharmaceutically acceptable salt thereof. In some embodiments, compound 87 or its pharmaceutically acceptable salt is useful in treating brain cancer, including, e.g., GBM.

In some embodiments, provided herein is compound 88 of Table 1, or a pharmaceutically acceptable salt thereof. In some embodiments, compound 88 or its pharmaceutically acceptable salt is useful in treating brain cancer, including, e.g., GBM.

In some embodiments, provided herein is compound 89 of Table 1, or a pharmaceutically acceptable salt thereof. In some embodiments, compound 89 or its pharmaceutically acceptable salt is useful in treating brain cancer, including, e.g., GBM.

In some embodiments, provided herein is compound 90 of Table 1, or a pharmaceutically acceptable salt thereof. In some embodiments, compound 90 or its pharmaceutically acceptable salt is useful in treating brain cancer, including, e.g., GBM.

In some embodiments, provided herein is compound 91 of Table 1, or a pharmaceutically acceptable salt thereof. In some embodiments, compound 91 or its pharmaceutically acceptable salt is useful in treating brain cancer, including, e.g., GBM.

In some embodiments, provided herein is compound 92 of Table 1, or a pharmaceutically acceptable salt thereof. In some embodiments, compound 92 or its pharmaceutically acceptable salt is useful in treating brain cancer, including, e.g., GBM.

In some embodiments, provided herein is compound 93 of Table 1, or a pharmaceutically acceptable salt thereof. In some embodiments, compound 93 or its pharmaceutically acceptable salt is useful in treating brain cancer, including, e.g., GBM.

In some embodiments, provided herein is compound 94 of Table 1, or a pharmaceutically acceptable salt thereof. In some embodiments, compound 94 or its pharmaceutically acceptable salt is useful in treating brain cancer, including, e.g., GBM.

In some embodiments, provided herein is compound 95 of Table 1, or a pharmaceutically acceptable salt thereof. In some embodiments, compound 95 or its pharmaceutically acceptable salt is useful in treating brain cancer, including, e.g., GBM.

In some embodiments, provided herein is compound 96 of Table 1, or a pharmaceutically acceptable salt thereof. In some embodiments, compound 96 or its pharmaceutically acceptable salt is useful in treating brain cancer, including, e.g., GBM.

In some embodiments, provided herein is compound 97 of Table 1, or a pharmaceutically acceptable salt thereof. In some embodiments, compound 97 or its pharmaceutically acceptable salt is useful in treating brain cancer, including, e.g., GBM.

In some embodiments, provided herein is compound 98 of Table 1, or a pharmaceutically acceptable salt thereof. In some embodiments, compound 98 or its pharmaceutically acceptable salt is useful in treating brain cancer, including, e.g., GBM.

In some embodiments, provided herein is compound 99 of Table 1, or a pharmaceutically acceptable salt thereof. In some embodiments, compound 99 or its pharmaceutically acceptable salt is useful in treating brain cancer, including, e.g., GBM.

In some embodiments, provided herein is compound 100 of Table 1, or a pharmaceutically acceptable salt thereof. In some embodiments, compound 100 or its pharmaceutically acceptable salt is useful in treating brain cancer, including, e.g., GBM.

Aspects of the present disclosure are directed to methods of treating brain cancer and/or solid tumors compounds in a subject in need thereof. The method includes administering a therapeutically effective amount of a histone deacetylase 7 (HDAC-7) inhibitor to the subject.

Another aspect of the present disclosure is directed to methods of treating glioblastoma multiforme in a subject in need thereof. The method includes administering a therapeutically effective amount of a histone deacetylase 7 (HDAC-7) inhibitor to the subject.

In some embodiments, the HDAC-7 inhibitor includes one or more of the compounds in Table 1 or a pharmaceutically acceptable salt thereof, or a pharmaceutical composition comprising one or more of the compounds in Table 1 or the pharmaceutically acceptable salt thereof.

In some embodiments, the HDAC-7 inhibitor includes one or more of TMP269, trichostatin A, vorinostat, or a pharmaceutically acceptable salt thereof, or a pharmaceutical composition that includes one or more of TMP269, trichostatin A, vorinostat, or the pharmaceutically acceptable salt thereof.

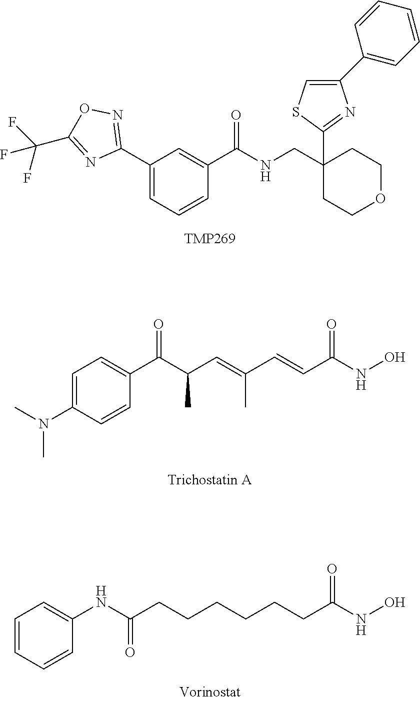 [SMILES: O=C(NCC1(c2nc(-c3ccccc3)cs2)CCOCC1)c1cccc(-c2noc(C(F)(F)F)n2)c1 ; CC(/C=C/C(=O)NO)=C\[C@@H](C)C(=O)c1ccc(N(C)C)cc1 ; O=C(CCCCCCC(=O)Nc1ccccc1)NO]

In some embodiments, the HDAC-7 inhibitor includes one or more siRNA or a pharmaceutically acceptable salt thereof, or a pharmaceutical composition that includes one or more of the siRNA or the pharmaceutically acceptable salt thereof.

In some embodiments, the siRNA comprises GACAAGAGCAAGCGAAGUG (SEQ ID NO: 1), GCAGAUACCCUCGGCUGAA (SEQ ID NO:2), GGUGAGGGCUUCAAUGUCA (SEQ ID NO: 3), or UGGCUGCUCUUCUGGGUAA (SEQ ID NO:4).

In some embodiments, the glioblastoma multiforme is present at least in part as a tumor.

In some embodiments, the HDAC-7 inhibitor is administered before, after or concurrently with surgical removal of at least part of the tumor.

In some embodiments, the HDAC-7 inhibitor is administered in an amount sufficient to cause one or more of the following: a decrease in tumor growth, a decrease in tumor cell proliferation, increased tumor cell apoptosis, inhibition of metastatic dissemination of the glioblastoma multiforme, improvement in subject survival, reduced ability of tumor cells to form colonies, or reduced ability of tumor cells to migrate.

In some embodiments, the HDAC-7 inhibitor is administered before, after or concurrently with the administration of an antibody therapy.

In some embodiments, the HDAC-7 inhibitor is administered before, after or concurrently with the administration of a radiation therapy.

Additional aspects of the present disclosure are directed to pharmaceutical compositions for treating brain cancer and/or solid tumors. In some embodiments, the composition includes one or more of the compounds in Table 1 or a pharmaceutically acceptable salt thereof.

Another aspect of the present disclosure is directed to pharmaceutical compositions for treating glioblastoma multiforme. In some embodiments, the composition includes one or more of the compounds in Table 1 or a pharmaceutically acceptable salt thereof.

Another aspect of the present disclosure is directed to pharmaceutical compositions for treating brain cancer and/or solid tumors. In some embodiments, the composition includes one or more siRNA or a pharmaceutically acceptable salt thereof. In some embodiments, the SIRNA comprises SEQ ID NO: 1, SEQ ID NO: 2, SEQ ID NO: 3, and SEQ ID NO: 4.

Another aspect of the present disclosure is directed to pharmaceutical compositions for treating glioblastoma multiforme. In some embodiments, the composition includes one or more siRNA or a pharmaceutically acceptable salt thereof. In some embodiments, the siRNA comprises SEQ ID NO: 1, SEQ ID NO: 2, SEQ ID NO: 3, and SEQ ID NO:4.

As used herein, “pharmaceutically acceptable salts” refers to an ionizable therapeutic agent that has been combined with a counter-ion to form a neutral complex. Lists of suitable salts are found, for example, in Remington's Pharmaceutical Sciences, 17th ed., Mack Publishing Company, Easton, Pa., 1985, p. 1418, Journal of Pharmaceutical Science, 66, 2 (1977), and “Handbook of Pharmaceutical Salts: Properties, Selection, and Use” (P. Henrich Stahl & Camille G. Wermuth (Eds.), VHCA & Wiley-VCH, 2002).

The terms “pharmaceutical” and “pharmaceutically acceptable” may refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

The terms “effective amount” or “therapeutically effective amount” refer to an amount, i.e. a dosage, of therapeutic agent administered to a subject (e.g., a mammalian subject, i.e. a human subject), either as a single dose or as part of a series of doses, which is effective to produce a desired therapeutic effect (e.g., effective for influencing, reducing or inhibiting the activity of or preventing activation of a kinase, or effective at bringing about a desired in vivo effect in an animal, preferably, a human, such as reduction in intraocular pressure).

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

The term “treatment” may refer to the application of one or more specific procedures used for the amelioration of a disease. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a therapeutic agent or a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset.

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

The brain cancer and/or solid tumor, glioblastoma multiforme therapies may further include any well-known therapies to treat cancer, including, but not limited to, surgical removal of the cancer, administration of chemotherapy, administration of radiation, administration of antibody therapies, and administration of anti-cancer drugs.

The term “chemotherapy” refers to the treatment of cancer or a disease or disorder caused by a virus, bacterium, other microorganism, or an inappropriate immune response using specific chemical agents, drugs, or radioactive agents that are selectively toxic and destructive to malignant cells and tissues, viruses, bacteria, or other microorganisms. Chemotherapeutic agents or drugs such as an anti-folate (e.g., methotrexate) or any other agent or drug useful in treating cancer, an inflammatory disease, or an autoimmune disease are preferred. Suitable chemotherapeutic agents and drugs include, but are not limited to, actinomycin D, adriamycin, altretamine, azathioprine, bleomycin, busulphan, capecitabine, carboplatin, carmustine, chlorambucil, cisplatin, cladribine, crisantaspase, cyclophosphamide, cytarabine, dacarbazine, daunorubicin, doxorubicin, epirubicin, etoposide, fludarabine, fluorouracil, gemcitabine, hydroxyurea, idarubicin, ifosfamide, irinotecan, liposomal doxorubicin, lomustine, melphalan, mercaptopurine, methotrexate, mitomycin, mitozantrone, oxaliplatin, paclitaxel, pentostatin, procarbazine, raltitrexed, steroids, streptozocin, taxol, taxotere, temozolomide, thioguanine, thiotepa, tomudex, topotecan, treosulfan, uft (uracil-tegufur), vinblastine, vincristine, vindesine, and vinorelbine.

As used herein, in such methods the term “biological sample” refers to a body fluid or tissue. The body fluid can include, without limitation, whole blood, serum, plasma, peripheral blood, synovial fluid, cerebrospinal fluid, saliva, urine, semen, or other fluid secretion. The term “tissue” can include, without limitation, bone marrow and lymph node, as well as samples of other tissues.

The present disclosure also provides pharmaceutical compositions comprising an effective amount of a compound of a HDAC-7 inhibitor disclosed herein, or a pharmaceutically acceptable salt thereof; and a pharmaceutically acceptable carrier. In certain embodiments, the disclosure also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

“Pharmaceutically acceptable carrier” means a carrier that is useful for the preparation of a pharmaceutical composition that is: generally compatible with the other ingredients of the composition, not deleterious to the recipient, and neither biologically nor otherwise undesirable. “A pharmaceutically acceptable carrier” includes both one and more than one carrier. Embodiments include carriers for topical, ocular, parenteral, intravenous, intraperitoneal intramuscular, sublingual, nasal, and oral administration. “Pharmaceutically acceptable carrier” also includes agents for preparation of aqueous dispersions and sterile powders for injection or dispersions.

Pharmaceutically acceptable carriers, adjuvants and vehicles that may be used in the pharmaceutical compositions of the present disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present disclosure include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product. Also, see, for example, Ansel's Pharmaceutical Dosage Forms and Drug Delivery Systems, Wolters Kluwer Health (11th ed. 2018).

In some embodiments, any one of the compounds and therapeutic agents disclosed herein are administered orally. Compositions of the present disclosure suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

The pharmaceutical compositions of the present disclosure may be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present disclosure is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Examples of useful dermatological compositions which can be used to deliver the compounds to the skin are known in the art; see for example, Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

The compounds and therapeutic agents of the present disclosure may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present disclosure provides an implantable drug release device impregnated with or containing a compound or a therapeutic agent, or a composition comprising a compound of the present disclosure or a therapeutic agent, such that said compound or therapeutic agent is released from said device and is therapeutically active.

### Dosages and Regimens

In the pharmaceutical compositions of the present disclosure, a compound of Formula (I) is present in an effective amount (e.g., a therapeutically effective amount).

Effective doses may vary, depending on the diseases treated, the severity of the disease, the route of administration, the sex, age and general health condition of the subject, excipient usage, and the possibility of co-usage with other therapeutic treatments such as use of other agents and the judgment of the treating physician.

In some embodiments, an effective amount of a HDAC-7 inhibitor can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of a HDAC-7 inhibitor is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

In some embodiments, provided herein are packaged dosage forms, comprising a container holding a therapeutically effective amount of an HDAC-7 inhibitor provided herein or its salt, and instructions for using the dosage form in accordance with one or more of the methods provided herein.

The present dosage forms and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, the described dosage forms can also be provided in kit form combined with other components necessary for administration of the material to the patient. For example, disclosed kits, such as for use in the treatments described herein, can further comprise, for example, administration materials.

The kits may be designed in various forms based on the specific deficiencies they are designed to treat.

The dosage forms provided herein may be prepared and placed in a container for storage at ambient or elevated temperature. This is beneficial because transportation of commercially viable dosage forms may benefit from stability at temperatures greater than those requiring refrigeration or sub-freezing environments during transportation and storage at the site of use.

When the dosage forms provided herein are stored in a polyolefin plastic container as compared to, for example, a polyvinyl chloride plastic container, discoloration of the dosage form may be reduced. Without wishing to be bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce adherence or adsorption of the active ingredient to the surface of the container, which could effectively dilute the concentration of active ingredient in the contained solution. Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include dosage forms that include one or more of the HDAC-7 inhibitors provided herein or its salt. The dosage forms provided herein may need storage lasting up to, or longer than, three months; in some cases up to, or longer than one year. The containers may be in any form suitable to contain the contents—for example, a bag, a bottle, or a box.

The following examples further illustrate embodiments of the present disclosure. However, they are in no way a limitation of the teachings or disclosure as described herein.

### EXAMPLES

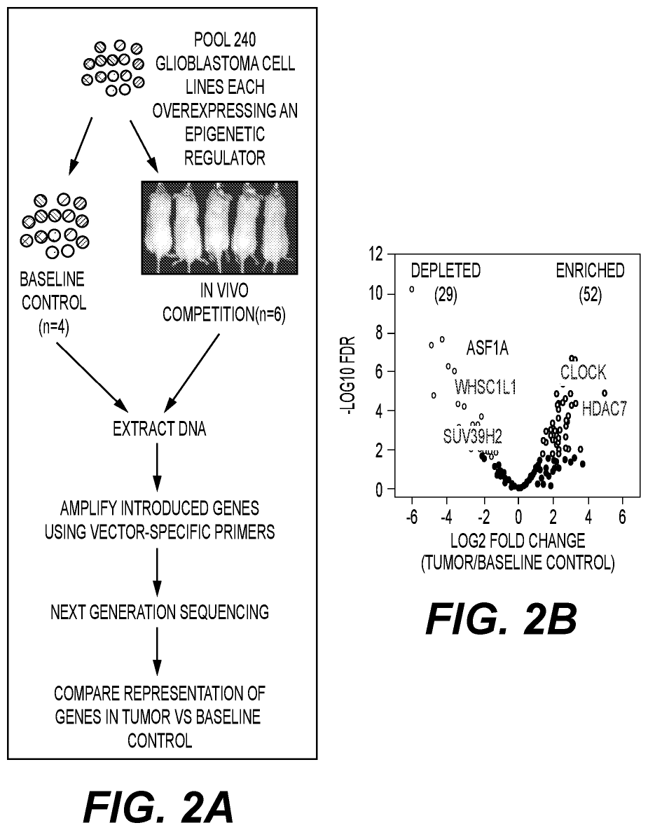

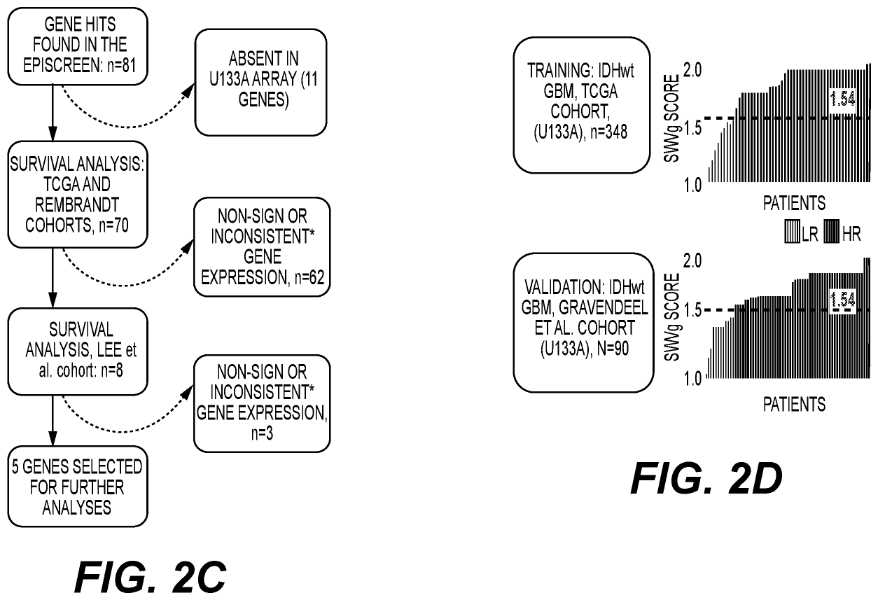

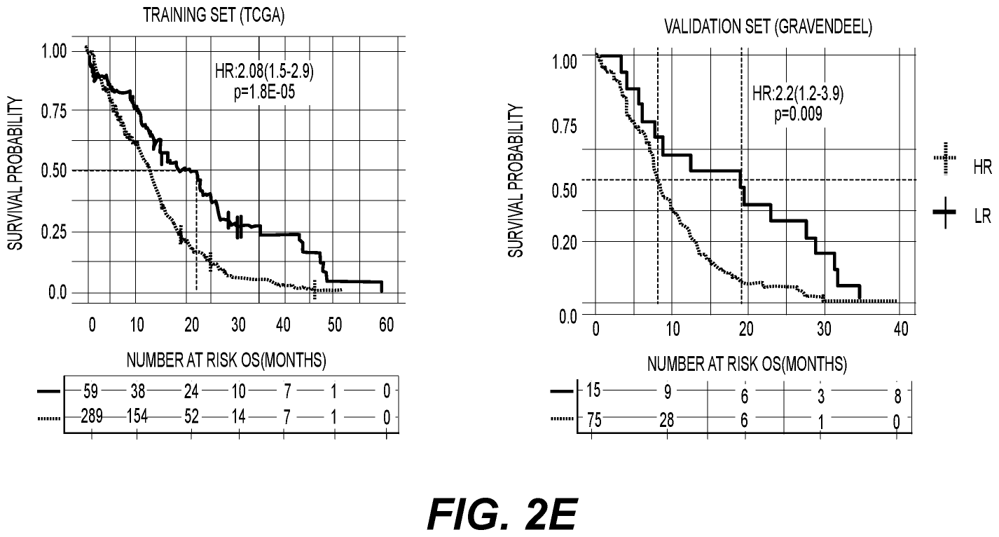

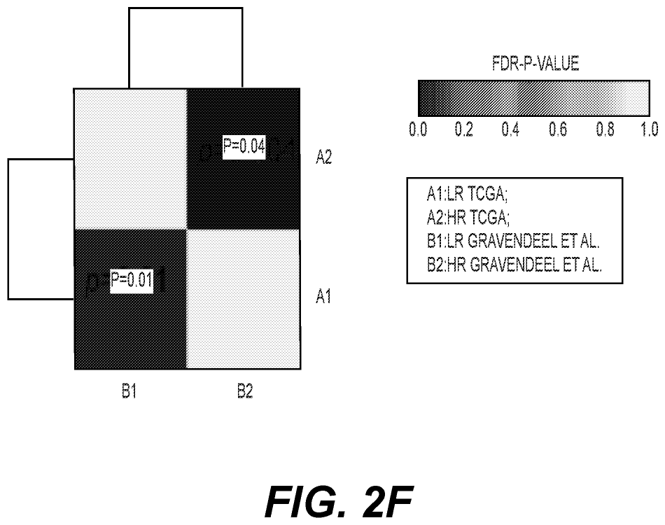

Through a viral library screen of all epigenetic regulators in a GBM human xenograft mouse model as well as in a GSC culture, epigenetic regulators were identified that, when overexpressed, would decease or increase the survival of a GBM mouse model or change the self-renewal phenotype of GSC in-vitro culture. In-vitro and in-vivo, five important epigenetic regulators were identified, which are HDAC-7, CLOCK, ASF1A, SUV39H2 and WHSC1L1, where the upregulation of HDAC-7 and CLOCK and the down regulation of ASF1A, SUV39H2 and WHSC1L1 presented a phenotype change in-vitro and decreased the survival of a GBM mouse model (FIG. 2B).

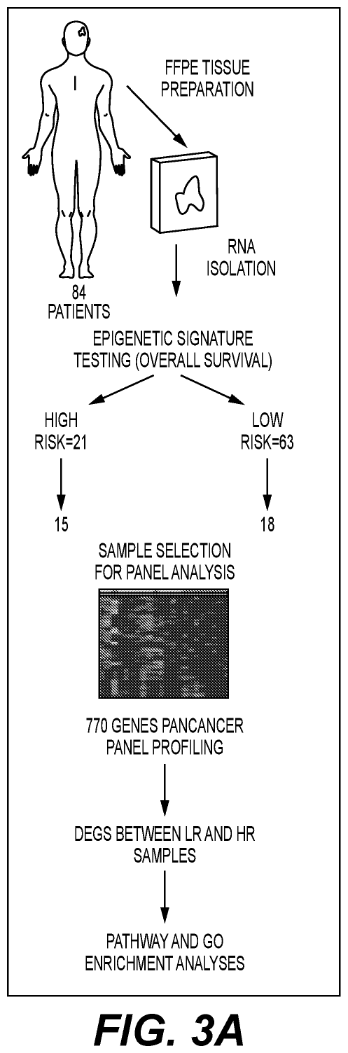

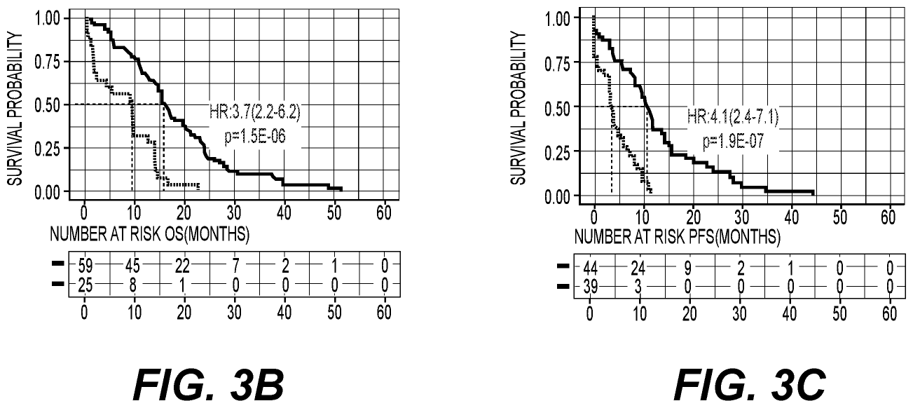

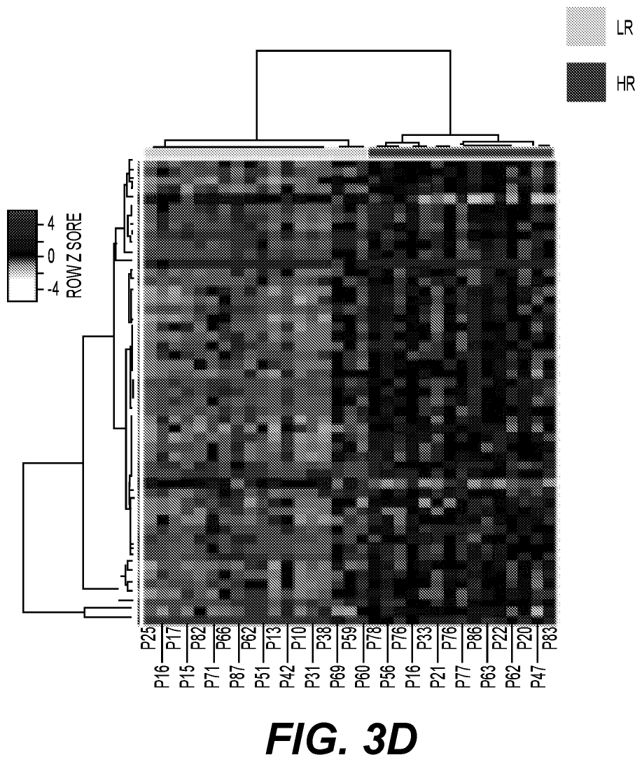

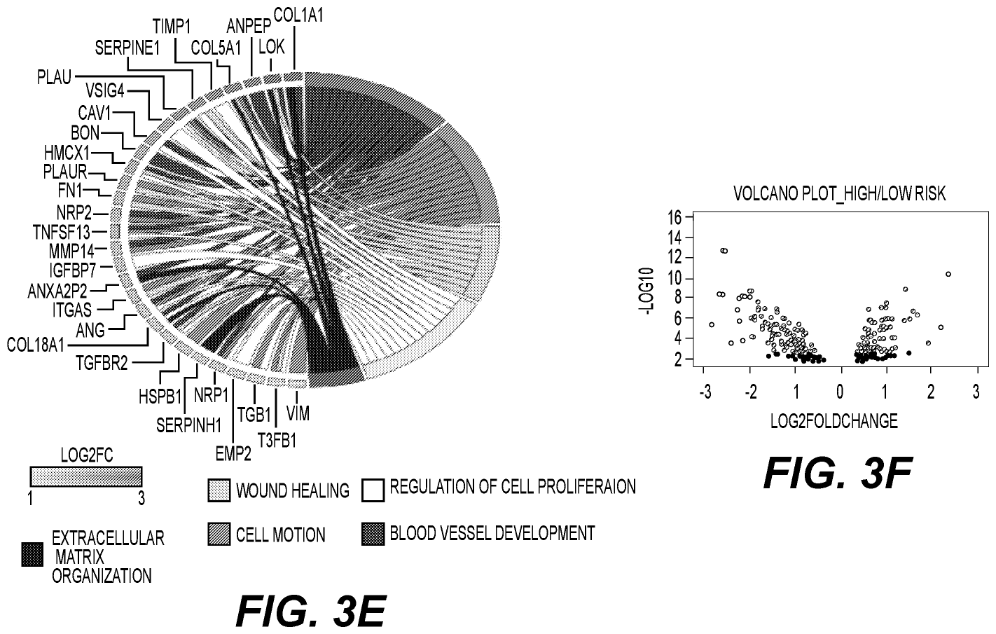

Next, RNA was collected from paraffin embedded samples from GBM patients and these RNA samples were ran against a PanCancer progression panel of 700 genes using the NonoString technology. Data for 33 patients were curated and categorized as high-risk (HR) and low-risk (LR) based on the risk derived from the expression of those five genes as epigenetic signatures. Differential gene expression analysis was applied on the HR versus LR group and 293/700 genes were found to be differentially expressed between the HR and the LR groups (FIG. 3D and FIG. 3F). The functional significance of the differentially expressed genes showed enrichment for biological processes of wound healing and cell proliferation (FIG. 3E). Looking into the patient's data, a Kaplan Meier survival analysis was performed based on the same HR/LR stratification using the log rank test and Gehan-Breslow-Wilcoxon method. A significant decrease in both the overall survival (FIG. 3B), and the progression-free survival (FIG. 3C) of these patients was found based on the risk derived from the expression of the five genes (HDAC-7, CLOCK, ASF1A, SUV39H2 and WHSC1L1) as epigenetic signatures.

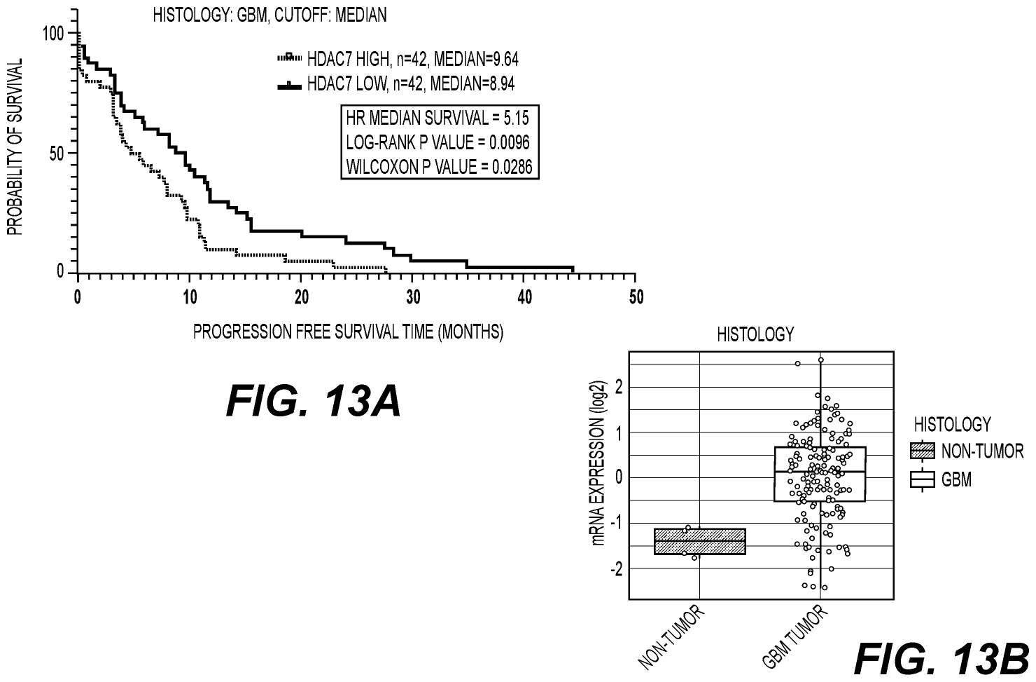

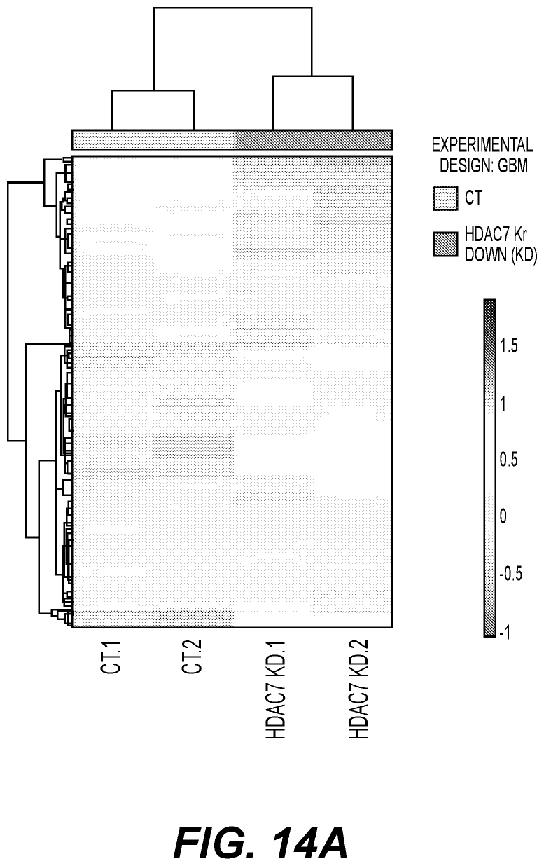

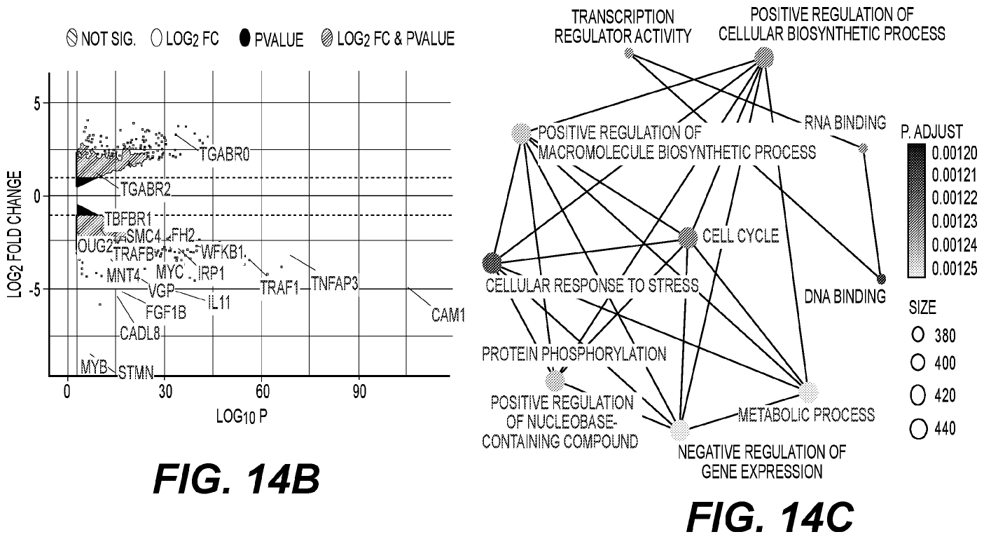

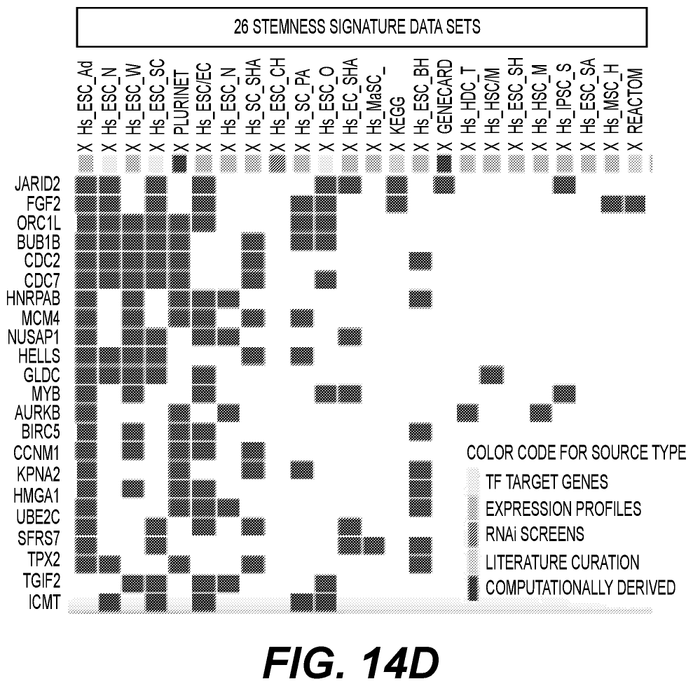

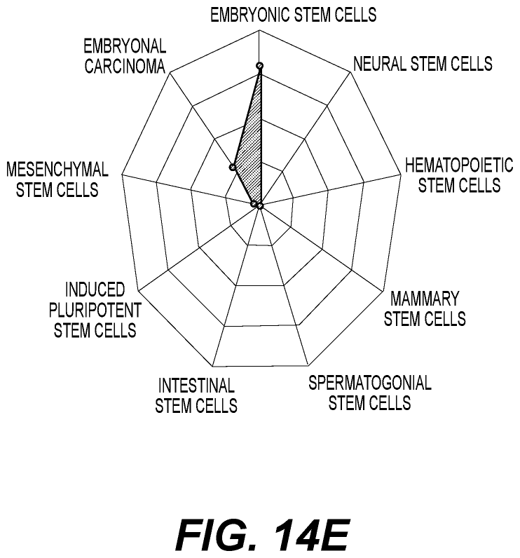

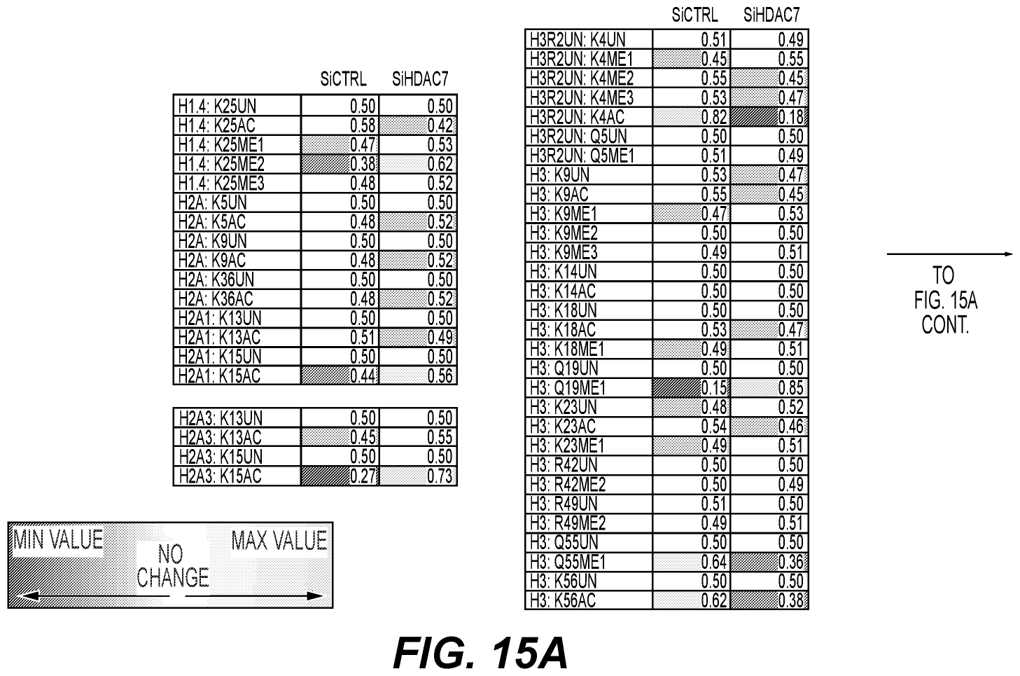

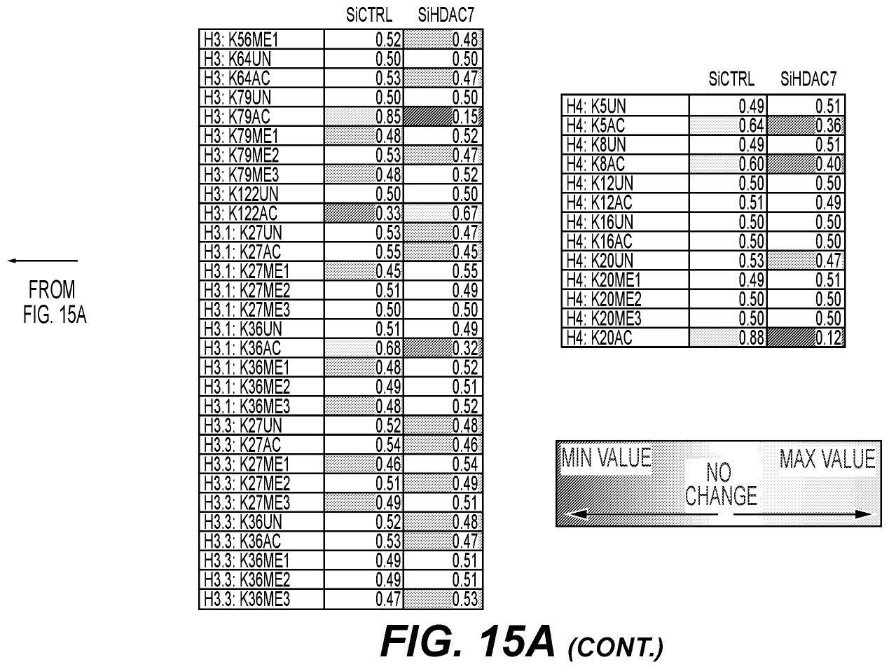

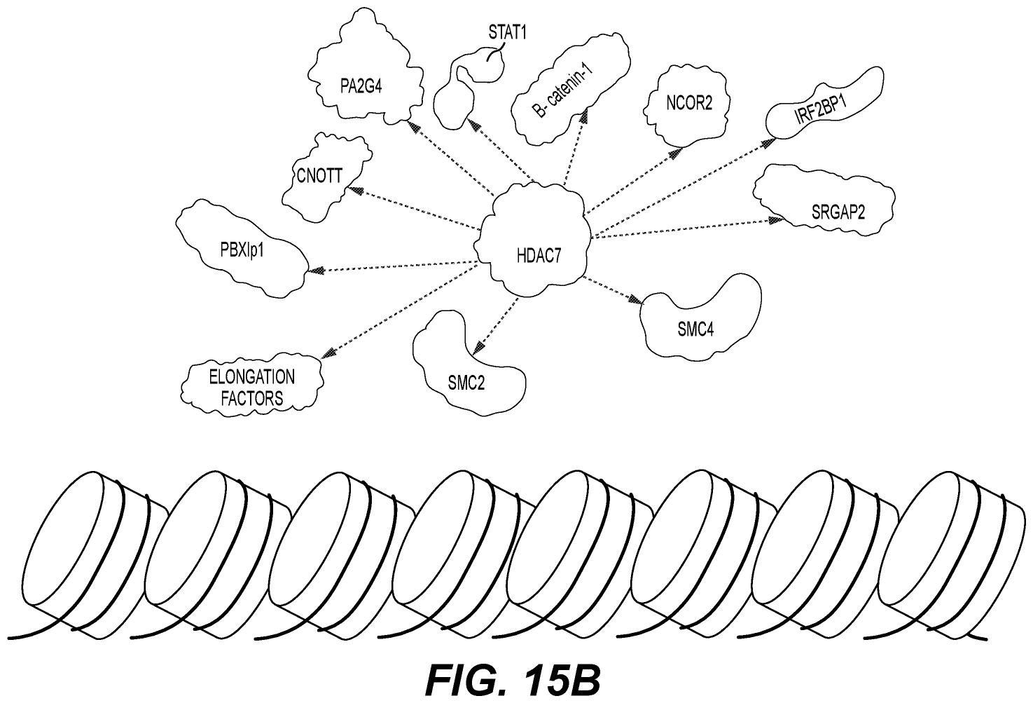

RNA was collected from paraffin embedded samples from GBM patients at Rhode Island Hospital (RIH) and survival analysis was performed according to HDAC-7 and mRNA expression. A significant decrease in the patient's survival for HDAC-7 high risk (HR) group as found (FIG. 13A). HDAC-7 mRNA expression in GBM was compared to the non-tumor samples from TCGA data using GlioVis webtool (Bowman, et al., R. L., Neuro. Oncol. 19, 139-141 (2017)) and it was found that HDAC-7 expression in GBM is exclusively high in GBM relative to non-tumor (FIG. 13A).

RNASeq was performed following HDAC-7 siRNA knock down in primary GSCs followed by transcriptomic analysis. Differential gene expression analysis with False discovery rate <0.01 between the HDAC-7 siRNA knock down and control siRNA cells was performed. For gene enrichment analysis, Gene Ontology and KEGG enrichment pathways were used (Smith. R. N. et al., Bioinformatics 28, 3163-3165 (2012)). Additionally, whether the genes that were downregulated by the HDAC-7 knockdown have role in stemness was checked by using the webtool Stem Checker (Pinto. J. P. et al., Nucleic Acids Res. 43, W72-W77 (2015)). A mass spectrometry was used on GSCs following HDAC-7 knock down to determine the role of HDAC-7 in regulating post translational modifications of histones on a global level. To elucidate the enzymatic de-acetylation effect of HDAC-7 inhibition on the global chromatin landscape HDAC-7 was knocked down in GSCs using siRNA, then a Mass Spectrometry analysis was performed using Mod Spec, that showed minimal de-acetylation on histone marks in GSCs. To unravel the protein partners for the HDAC7 in the GSC nucleus, Rapid-immunopreciptation-rnass-spectrometry-of-endogenous-protein (RIME) was performed on the GSCs using an HDAC-7 antibody.

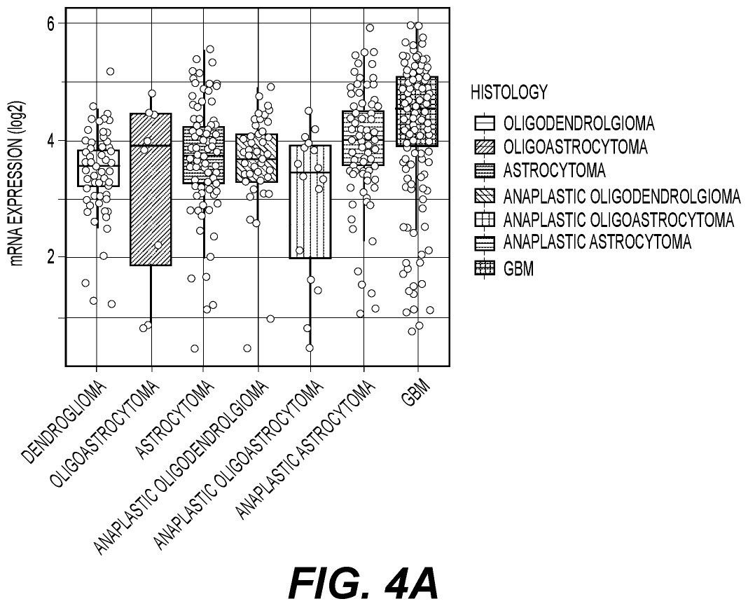

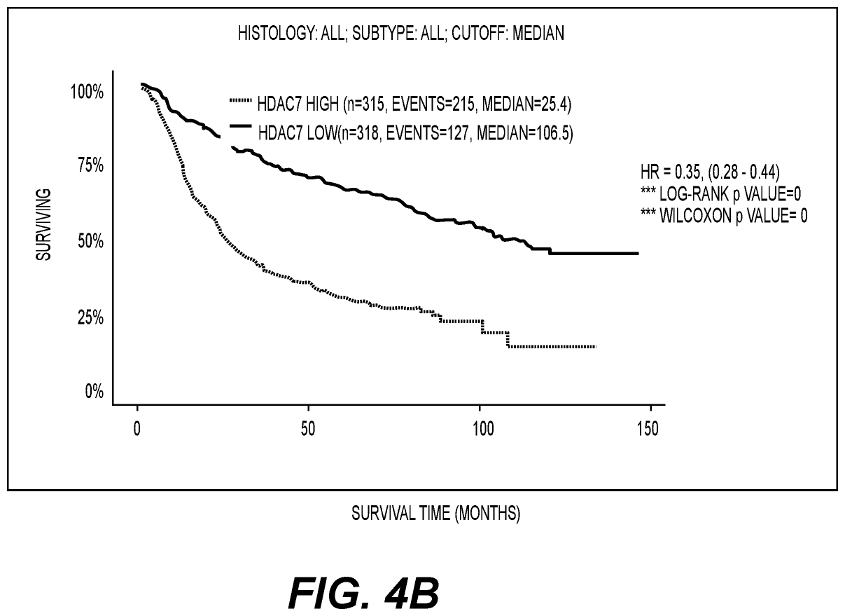

HDAC-7 was examined as one of the two epigenetic regulators that were upregulated in the viral screens, as well as for presenting a new target in GBM. On analyzing data from the Chinese Glioma Genome Atlas (CGGA) using GlioVis webtool6, the HDAC-7 mRNA expression in GBM was compared to other forms of malignant gliomas and it was found that HDAC-7 expression in GBM was the most pronounced expression among all other glial tumors (FIG. 4A).

Survival analyses were then performed on two GBM patient cohorts (HDAC-7 high- and low-expression patients), where patient were divided into these two groups based on the HDAC-7 expression as well as the maximally ranked statistics using the log rank test as well as Gehan-Breslow-Wilcoxon method. HDAC-7 expression positively correlated with the decrease of the overall survival of GBM patients (FIG. 4B).

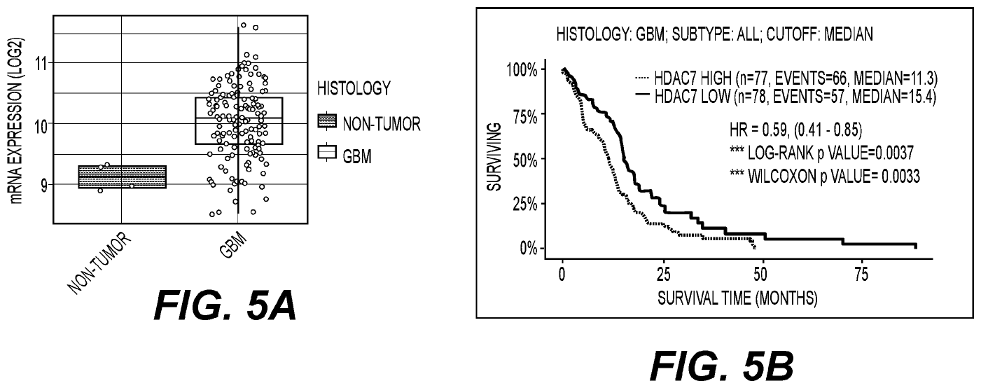

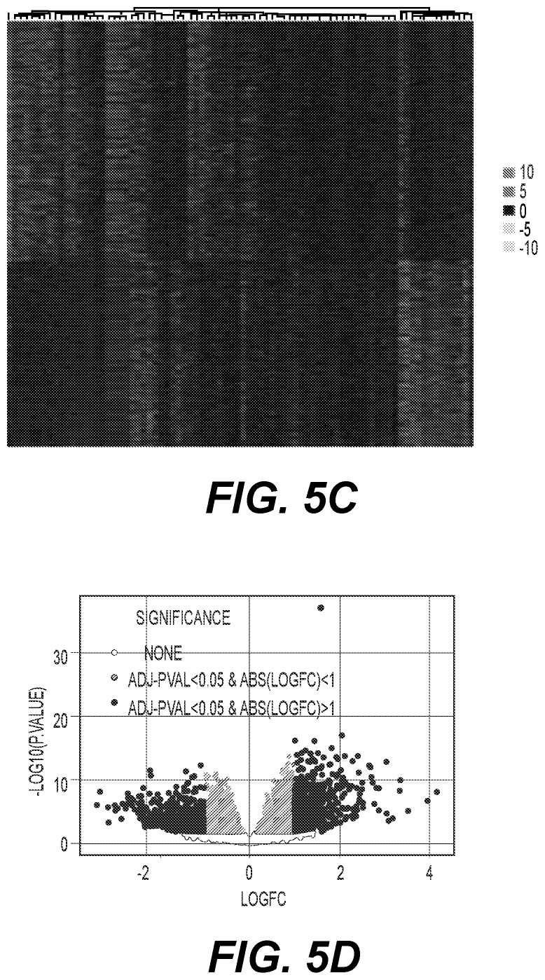

Similarly, on analyzing data from The Cancer Genome Atlas (TCGA), the HDAC-7 mRNA expression in GBM was compared to the non-tumor samples and it was found that HDAC-7 expression in GBM was exclusively high in GBM relative to non-tumor (FIG. 5A). Also, survival analysis were performed on two GBM patient cohorts (HDAC-7 high- and low-expression patients), with patients divided into these two groups according to the HDAC-7 expression as well as the maximally ranked statistics using the log rank test as well as Gehan-Breslow-Wilcoxon method. This showed that HDAC-7 expression is positively correlated with the decrease of the overall survival of the GBM patients (FIG. 5B). Moreover, a differential gene expression analysis was performed on the RNASeq data from TCGA and it was found that nearly 1600 of genes that were differentially expressed that were clustered into two distinct categories based on the HDAC-7 expression level (FIG. 5C) with the fold change in the differential expression is up to “4” in the upregulation and up to “−2.5” in the downregulation (FIG. 5D). These data from CGGA and TCGA supports a conclusion that HDAC-7 plays a crucial role in GBM pathology.

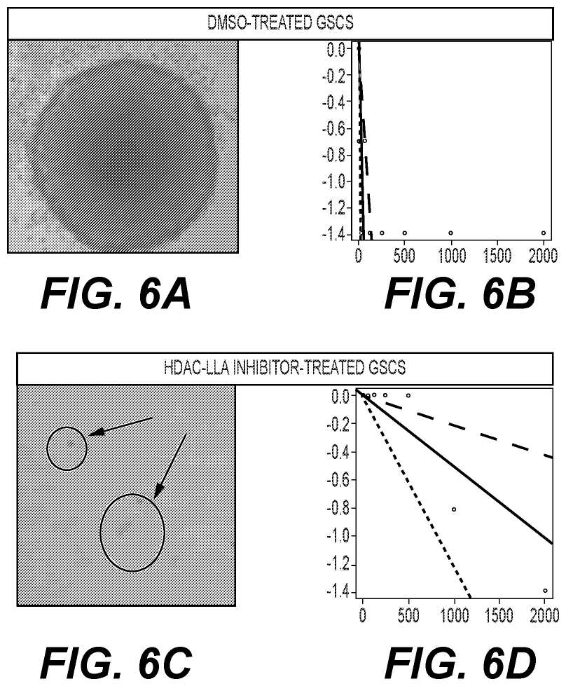

To validate whether HDAC-7 inhibition would present a phenotype change in primary GSCs, its effect on modifying the self-renewal of GSCs was examined, and since there is no small molecule drug currently available as a selective HDAC-7 inhibitor, the class IIa HDAC inhibitor, TMP269, that inhibits HDAC-4,5,7,9, was used. The two-week limiting dilution assay “LDA” that tests the frequency of self-renewing cells within a population was applied. Cells received the inhibitor versus DMSO-treated control cells, and a significant decrease was observed in the self-renewal of the TMP269 drug-treated cells in, which is evaluated as the ability of the GSCs to form neuro-spheres and through an online algorithmic scoring system. Moreover, the same significant result was observed when testing GSCs from different genotypes (FIGS. 6A-6D).

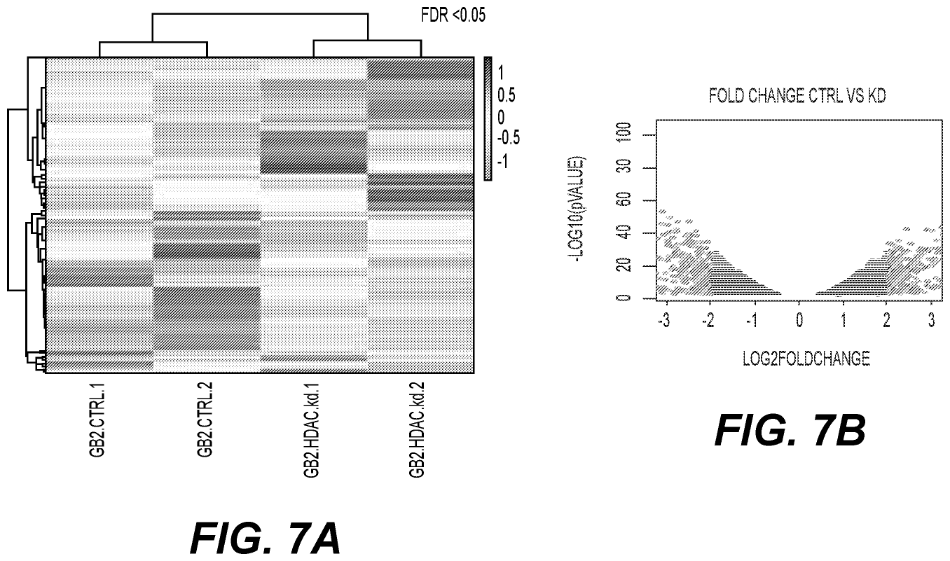

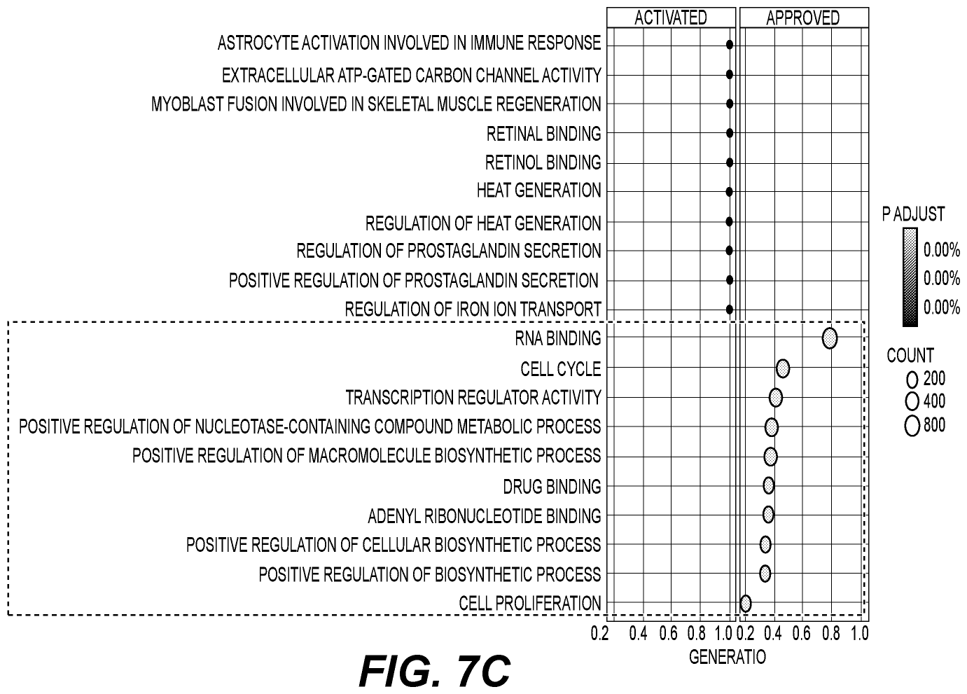

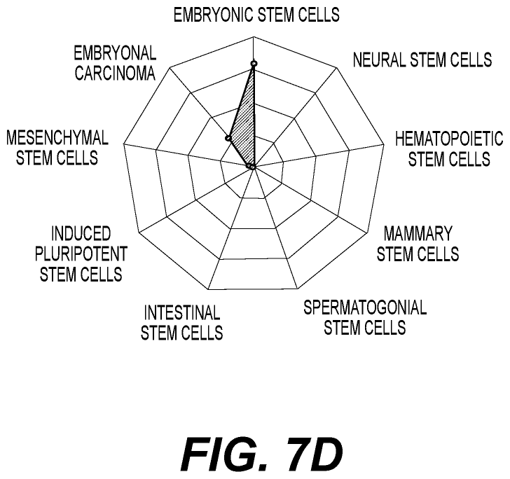

To test the genetic effect of HDAC-7 selective inhibition on GSCs, HDAC-7 in GSCs was knocked down using siRNAs (SEQ ID NOs: 1-4) and then transcriptomic analysis was applied using RNASeq. Differential gene expression analysis with less than 0.05 False discovery rate (FDR) showed that 6270 genes were differentially expressed between the HDAC-7 siRNA knock down and the SiRNA negative control with up to three-fold changes in the up- and the down-regulated genes (FIGS. 7A-7B). To understand the functional significance of those genes, gene enrichment analysis was applied on the differentially expressed genes using different tools for gene enrichment including Gene Ontology and KEGG enrichment pathways. It was found that the enriched pathways that were mainly suppressed were for biological processes for cell cycle activities and cell division (FIG. 7C). Additionally, whether the genes that were downregulated by the HDAC-7 knockdown have a role in stemness was checked by using the webtool Stem Checker. It was found that 653 genes out of 3150 downregulated genes have role in stemness, with those genes closer to the embryonic stem cells' and embryonal carcinoma's phenotype (FIG. 7D).

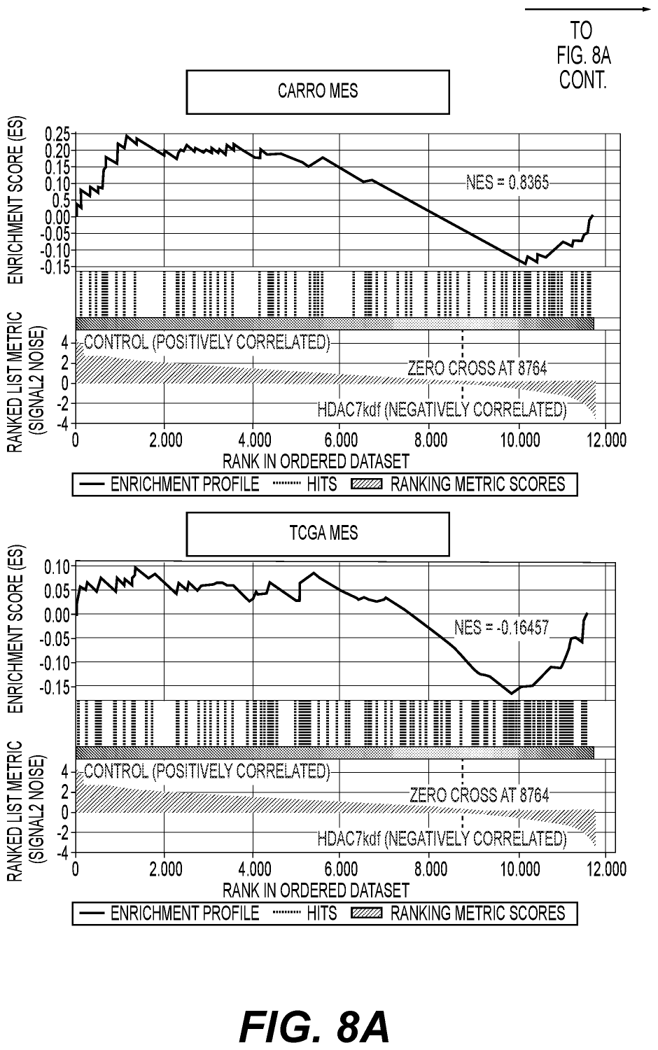

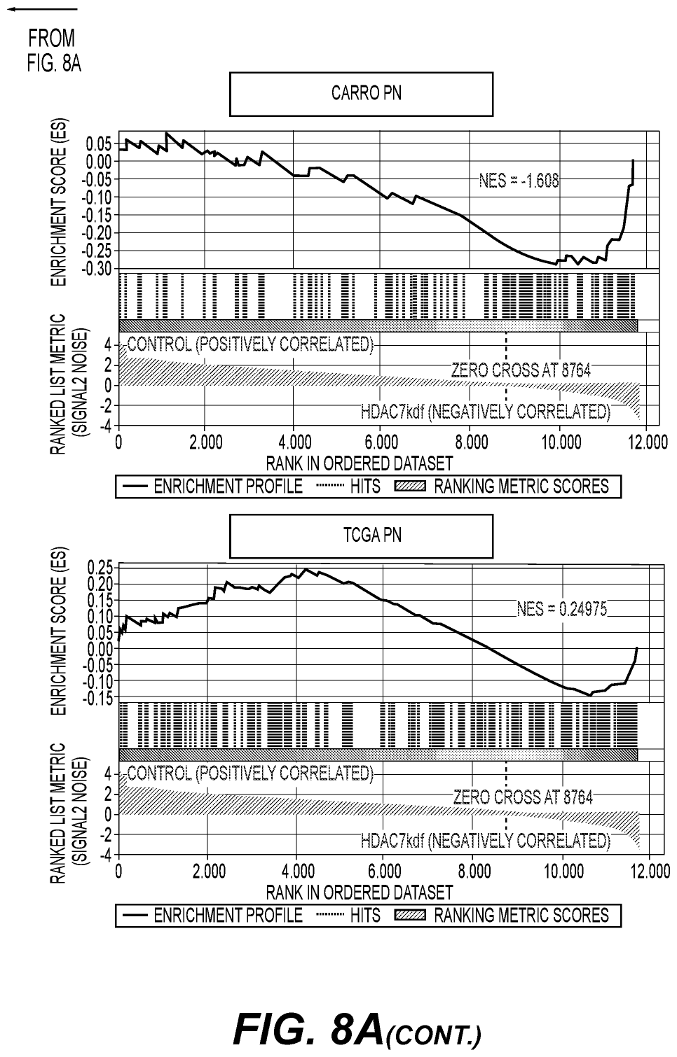

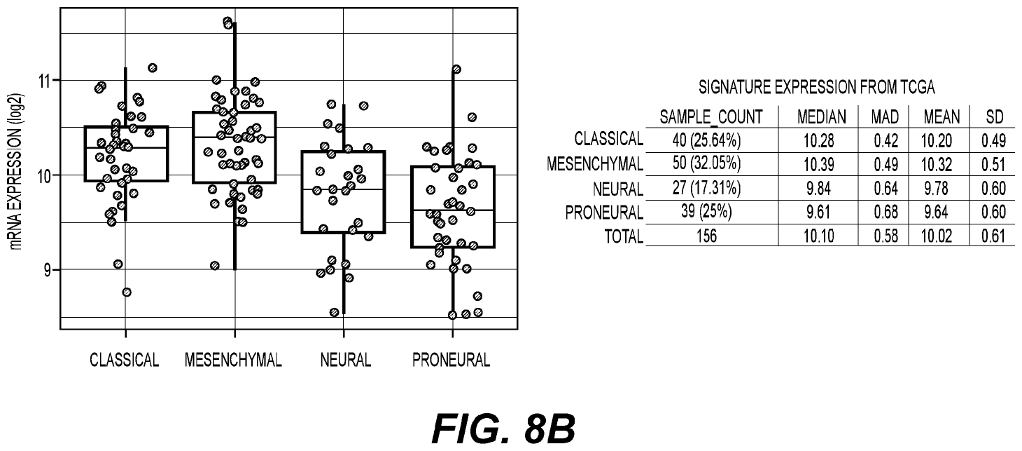

Like in many other cancers where the mesenchymal to epithelial phenotype of cells are usually associated with less aggressive tumors, the GBM also presents the same feature. In order to understand if HDAC-7 inhibition would have a role in this phenotypic transition, the Gene Set Enrichment Analysis webtool GSEA for the Broad Institute was used, which was supplied with each of the mesenchymal and proneural signature phenotypes gene set classifier according to TCGA13 and Carro signature stratification. By calculating the enrichment score according to the rank in each gene set, it was seen that HDAC-7 inhibition inhibits both the mesenchymal and the proneural gene set signatures (FIG. 8A). Also, to confirm these results, the expression of the signature genes of the various GSCs' subtypes in the publicly available data from TCGA was examined using GlioVis webtool6, and it was found that the four subtypes of GSCs present close proportions of HDAC-7 mRNA expression (FIG. 8B). This data supports a position that HDAC-7 inhibition could be suppressing all GSCs subtypes with its pathway not in the mesenchymal to proneural differentiation.

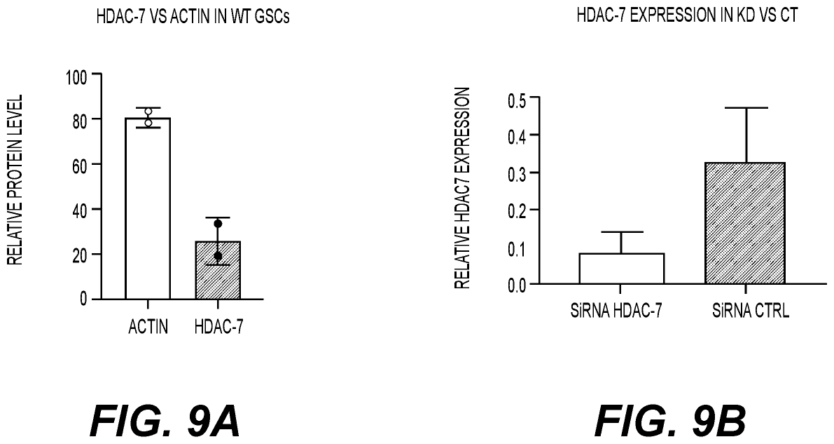

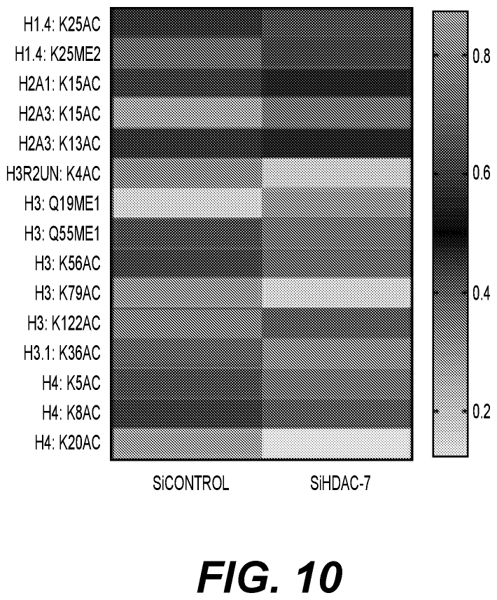

The interactome of HDAC-7 is not understood. To elucidate the effect of HDAC-7 inhibition on the global chromatin landscape, HDAC-7 was knocked down in GSCs using siRNA, validated by Western blot with an average of 73.4% protein inhibition (FIG. 9A). It was noted that the protein level in HDAC-7 in GSCs' lysates is relatively low compared to a housekeeping gene (FIG. 9B). A mass spec using Mod Spec technique was then applied on the HDAC-7 knockdown GSCs to determine the post-translational histone modification targets for HDAC-7 on a global level. Interestingly, the knock down showed a lot of histone modifications compared to the control, these changes include decrease or increase in the acetylation and the methylation of the lysine, arginine, and glutamine amino acids at the N-terminal histone tails. Out of more than 80 histone modifications that were tested in the Mod Spec, significant changes were found in the relative abundance of mainly 15 histone modifications between the knockdown and the control. Of particular interest was the relative increase in the acetylation of H3: K122AC in the HDAC-7 knockdown (FIG. 10). Recently, it was demonstrated that H3K122ac is sufficient to stimulate transcription, with the mutation of H3K122 disrupting the transcriptional activation, which was shown to be a direct effect of H3: K122AC on histone-DNA binding.

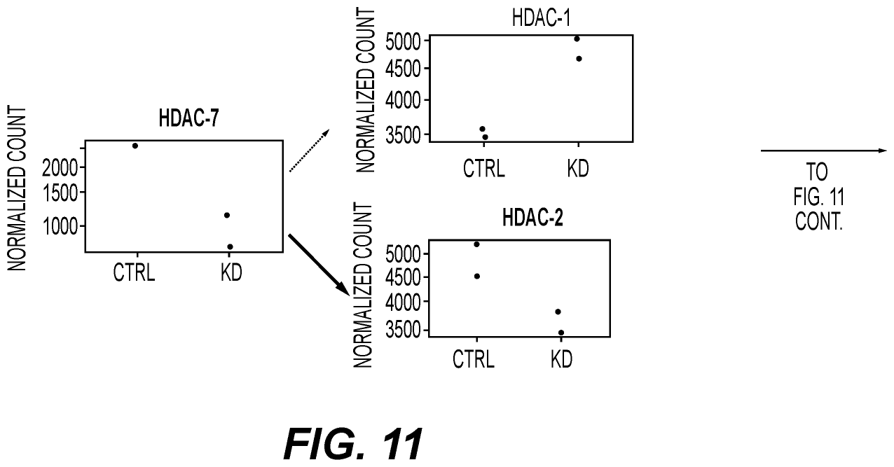

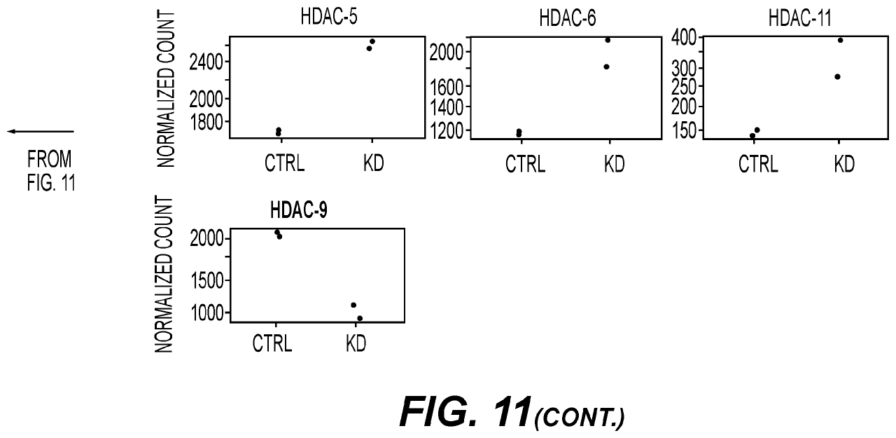

On the transcriptomic level, the significant effect of inhibiting HDAC-7 without the influence of other HDACs is seen (FIG. 7). From the RNASeq data it was found that knocking down HDAC-7 upregulated each of HDAC-1, -5, -6, and -11, and on the other hand downregulated HDAC-2, and -9 (FIG. 11). Thus, without being bound by theory, this could explain the increase in the acetylation in many PTM targets after HDAC-7 siRNA inhibition. Among the histone targets that showed a decrease in the acetylation, was that for H4: K20AC in the HDAC-7 knockdown. H4: K20AC is a unique acetylation mark associated with gene repression with H4: K20AC being enriched around TSSs of minimally expressed and silent genes. H4K20 may be acylated in highly active genes while the acetylation to be related with lower expressed genes. Another target is H3R2UN: K4AC.

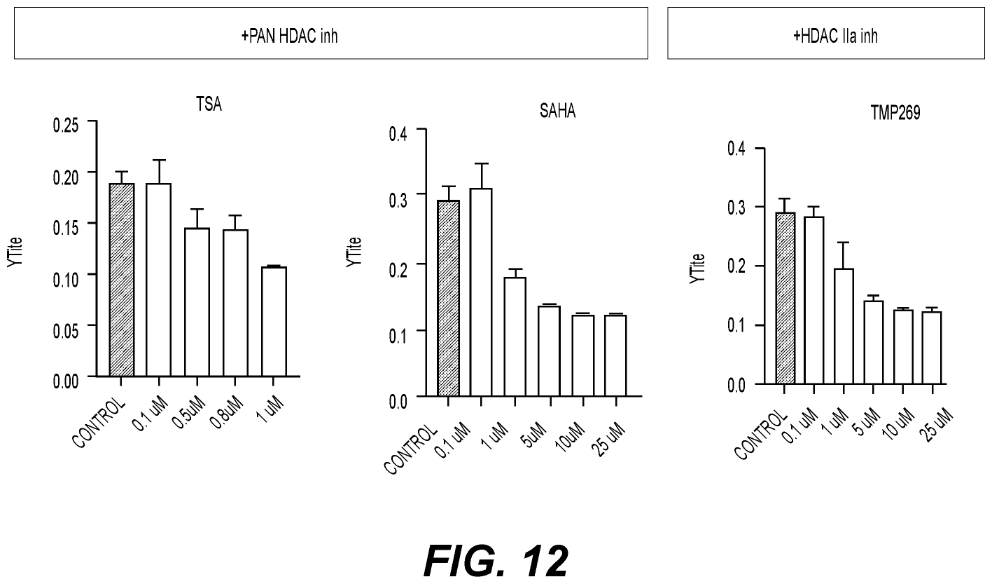

It has been found that HDAC class IIa inhibitor (TMP269) decreases the GSCs' viability by the same inhibitory trend seen from the pan HDAC inhibitor “Vorinostat”, which is an FDA approved drug as anticancer (FIG. 12). Preliminary results from the phenotypic (FIGS. 6 and 12) and genotypic experiments (FIG. 7) suggest that narrowing the spectrum for HDACs gives a comparable inhibitory effect to the pan HDACs, thus less off-target and side effects. HDAC-7 has been crystalized, and like other members of class I, II, and IV, HDAC-7 binds to the substrates through a zinc mediated charge relay system. Class IIa HDAC has another specific zinc-binding motif adjacent to the active site, which directs the recognition and protein-protein interaction. However, there is remarkable structural diversity in HDAC-7 found in loop regions around the active site entrance that poses a challenge to generation of inhibitors.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A compound, selected from: 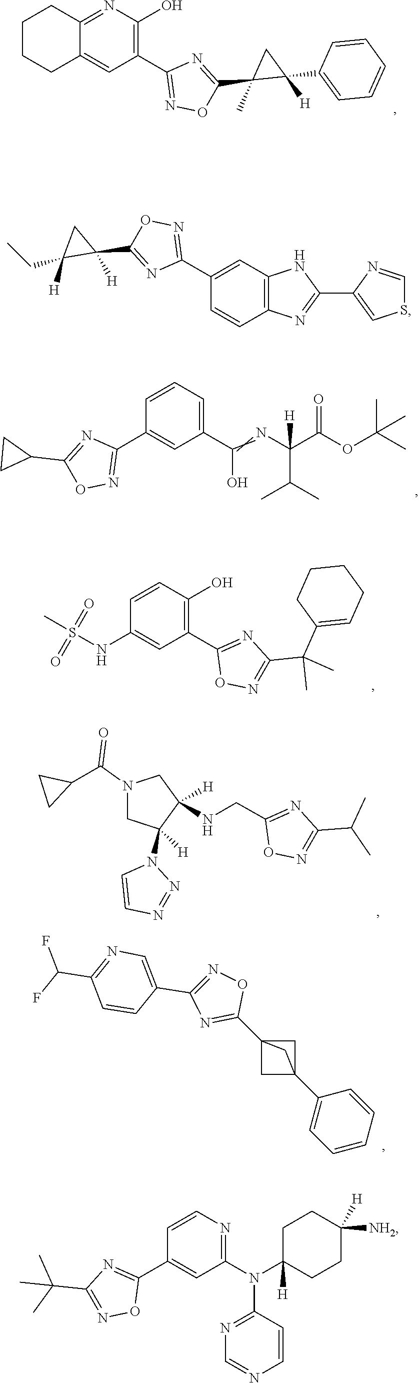 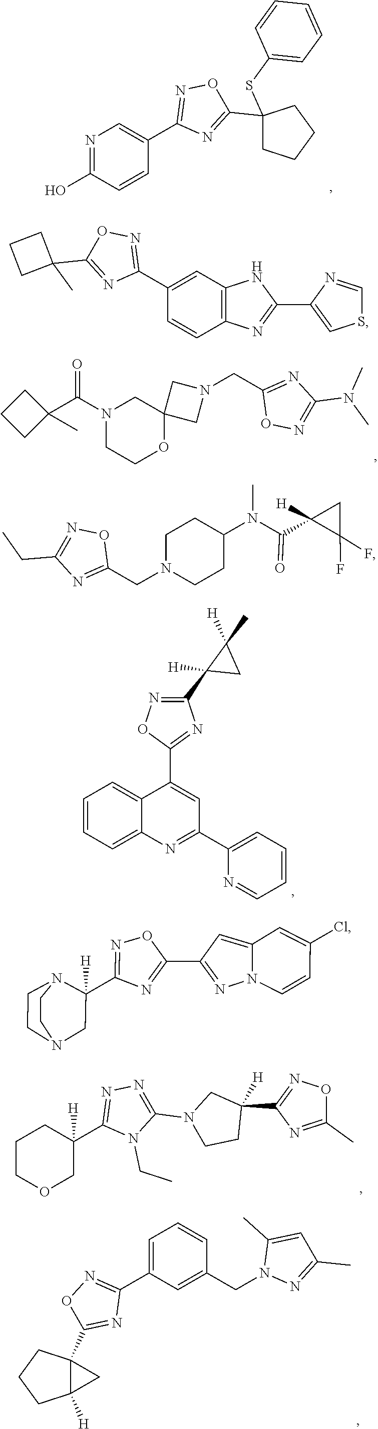 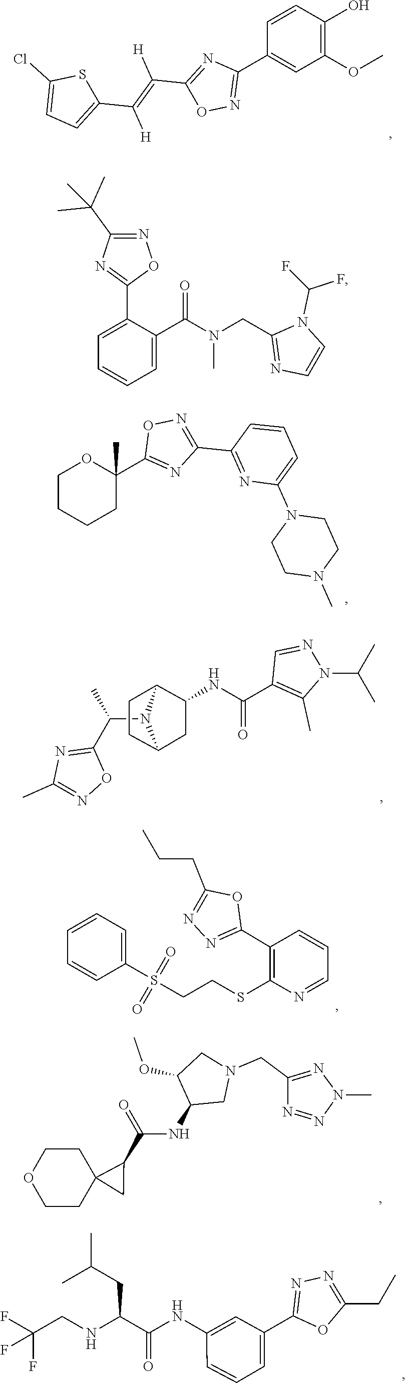 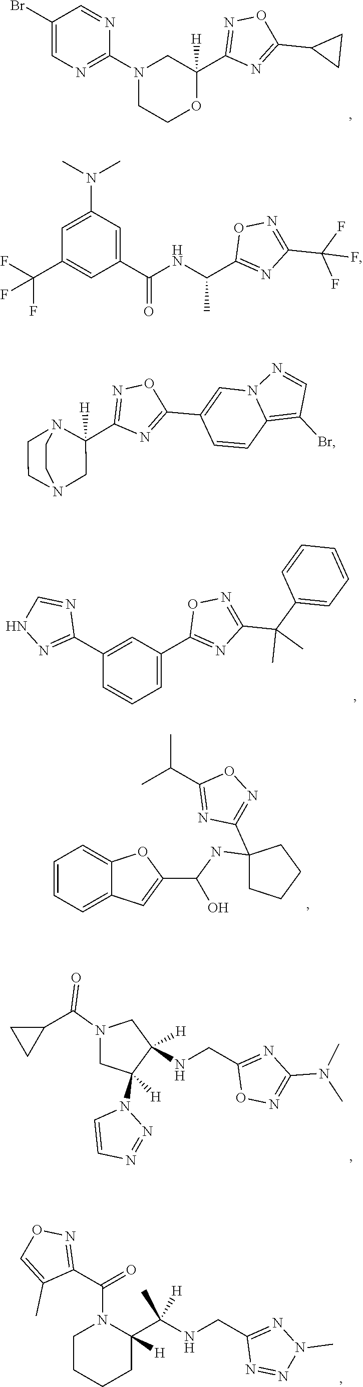 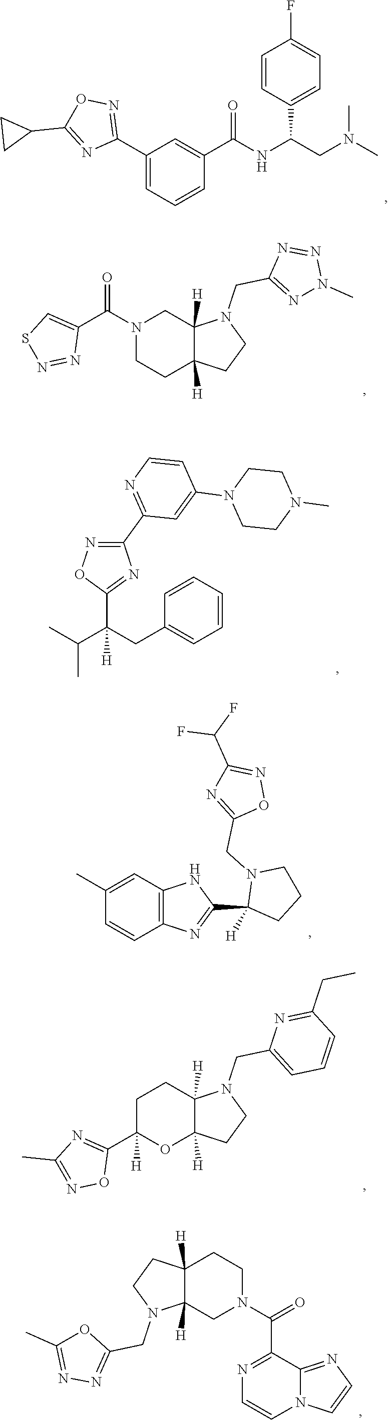 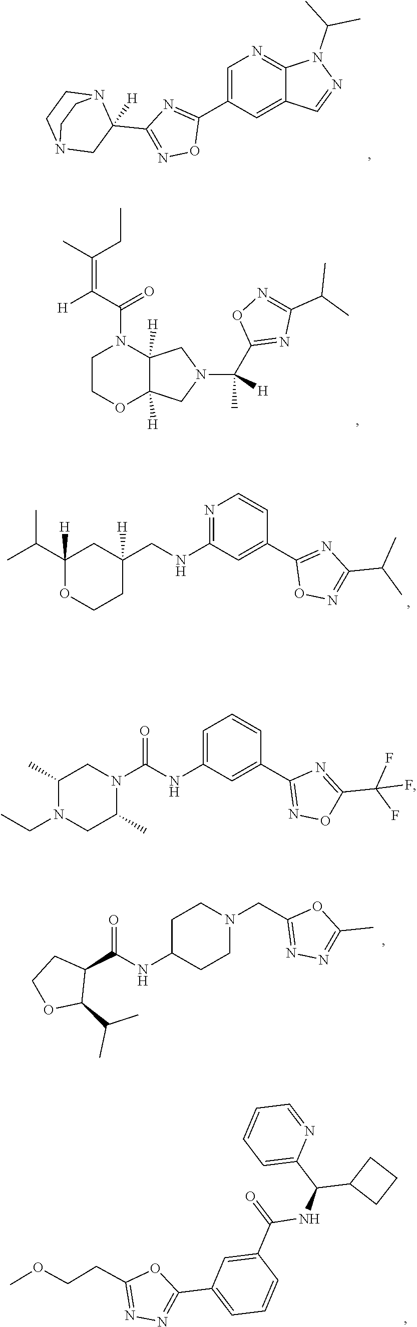 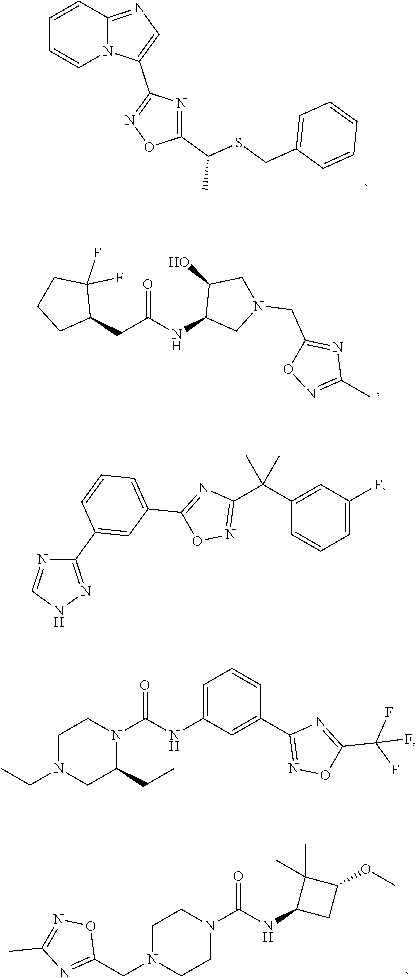 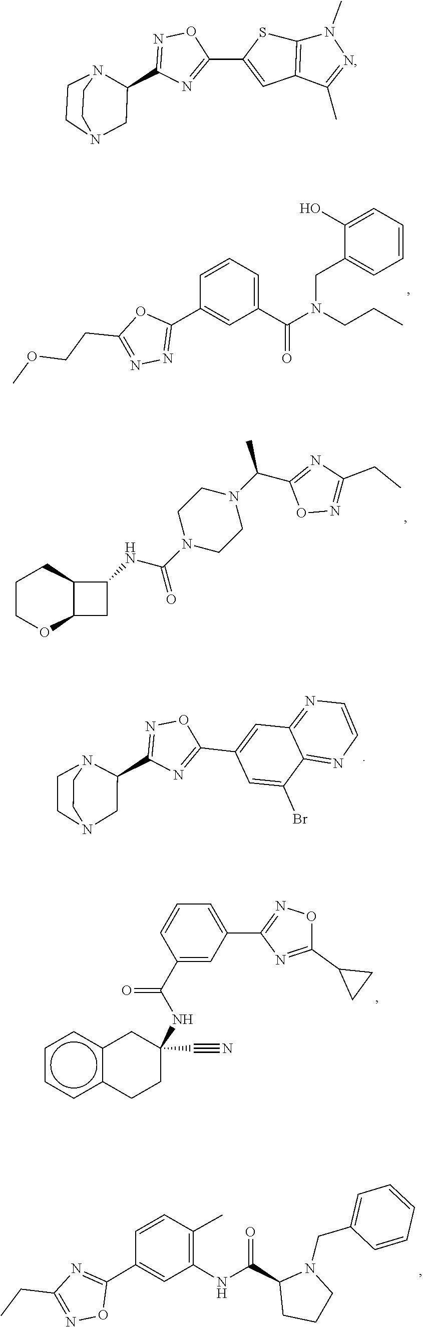 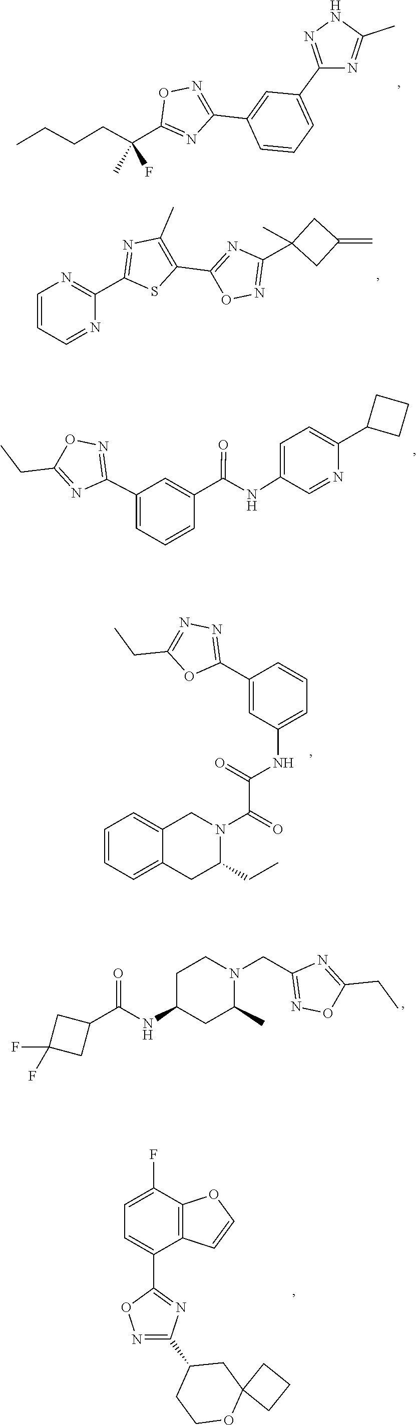 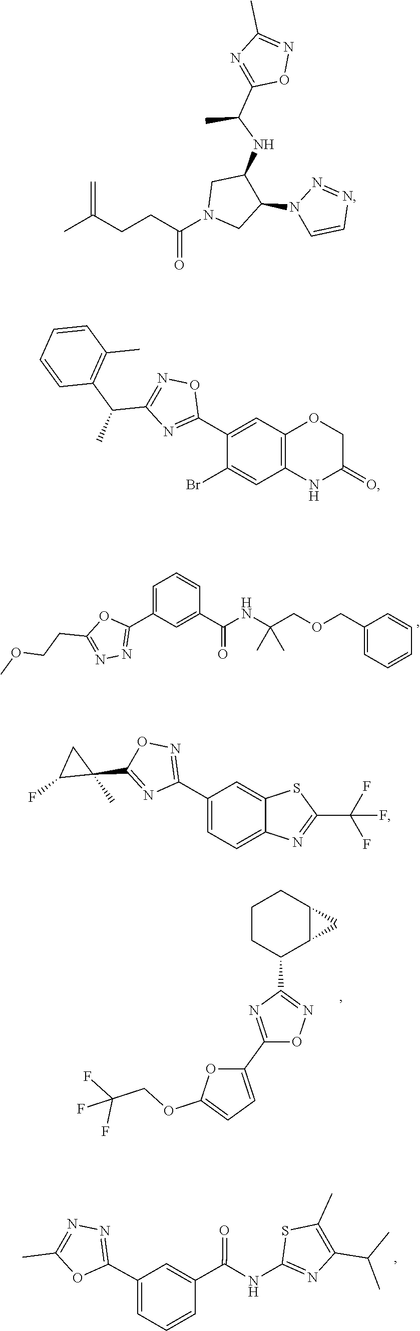 [SMILES: C=C(C)CCC(=O)N1C[C@@H](N[C@@H](C)c2nc(C)no2)[C@@H](n2ccnn2)C1 ; Cc1ccccc1[C@@H](C)c1noc(-c2cc3c(cc2Br)NC(=O)CO3)n1 ; COCCc1nnc(-c2cccc(C(=O)NC(C)(C)COCc3ccccc3)c2)o1 ; C[C@@]1(c2nc(-c3ccc4nc(C(F)(F)F)sc4c3)no2)C[C@H]1F ; FC(F)(F)COc1ccc(-c2nc([C@@H]3CCC[C@@H]4C[C@@H]43)no2)o1 ; Cc1nnc(-c2cccc(C(=O)Nc3nc(C(C)C)c(C)s3)c2)o1] 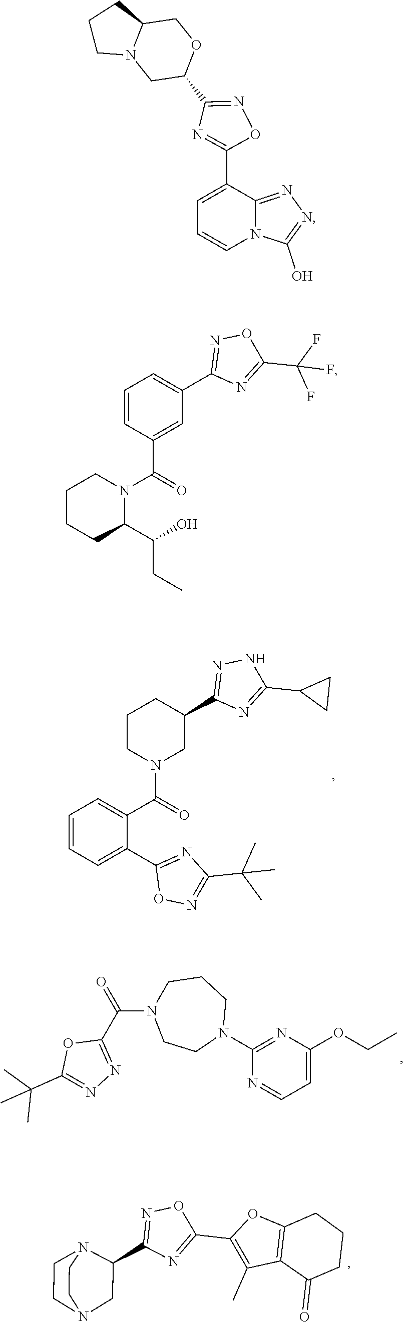 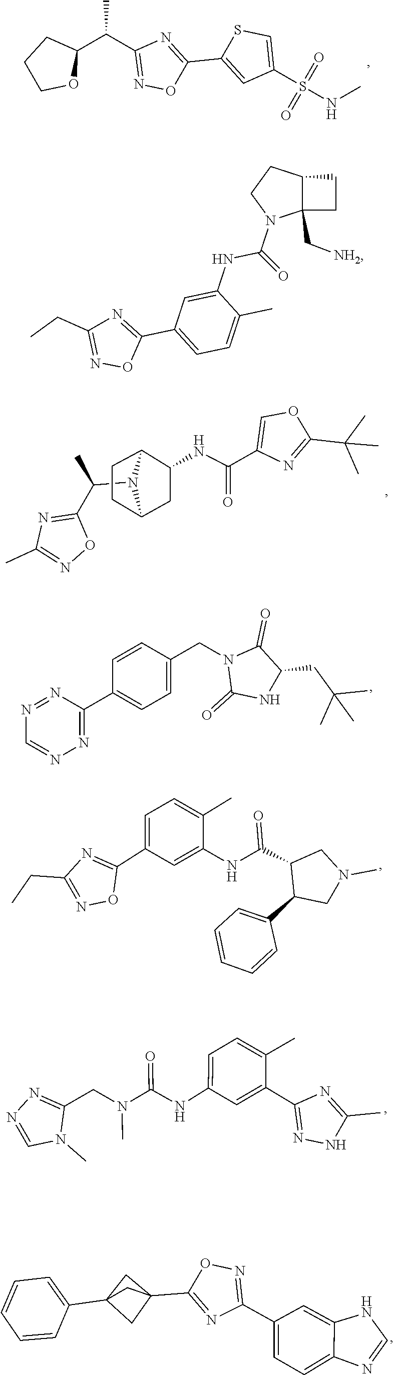 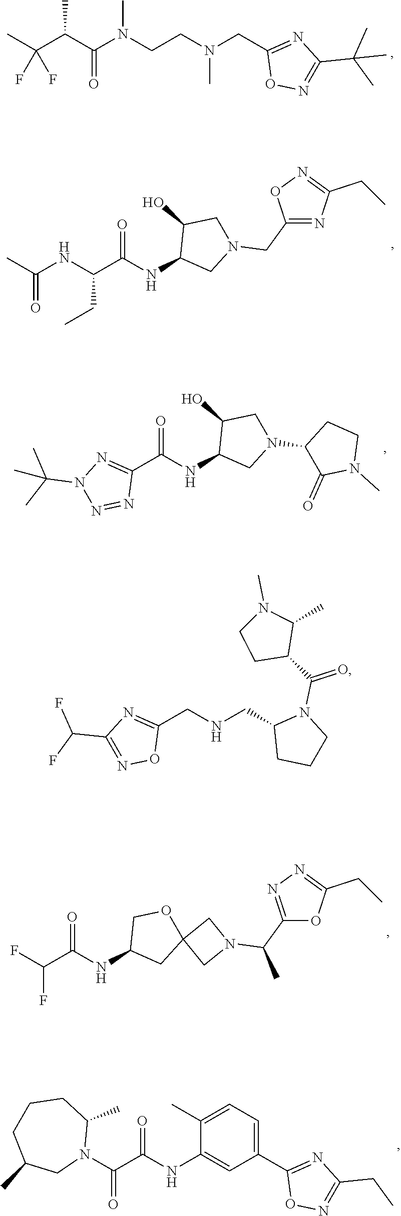 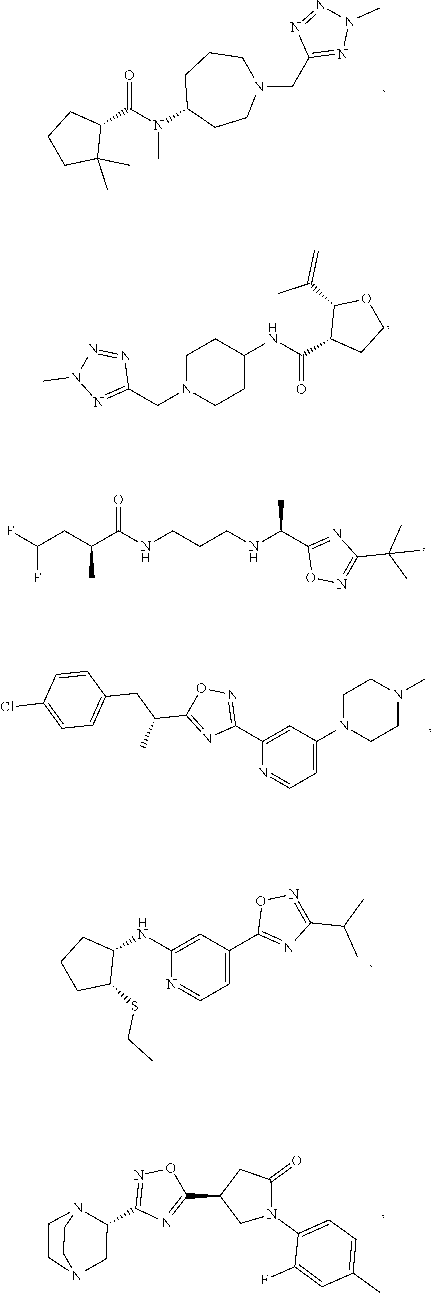 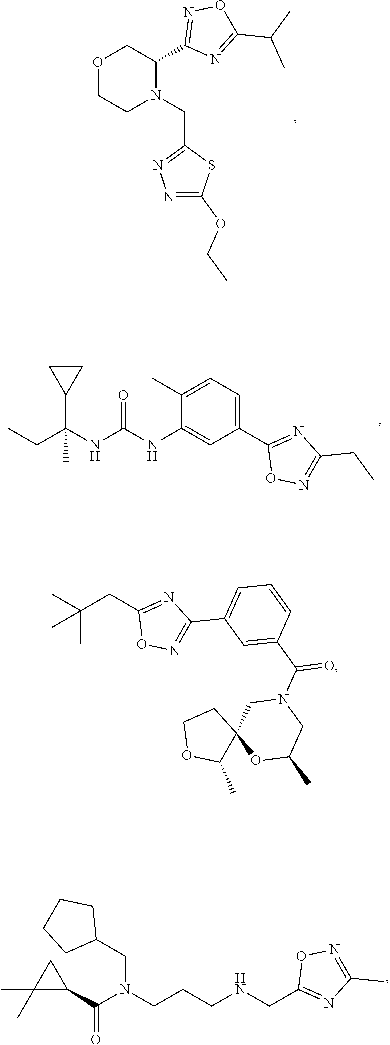 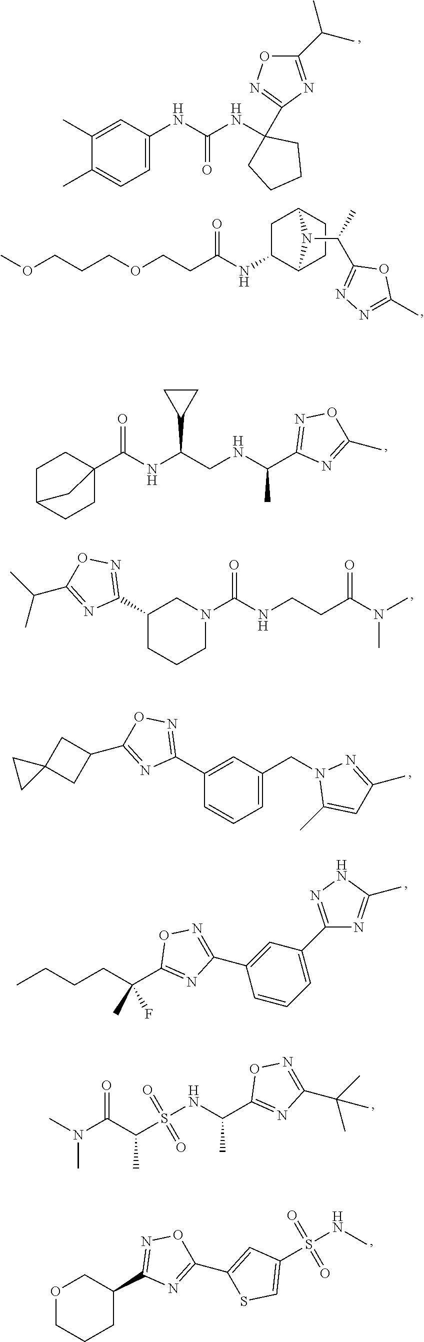
or a pharmaceutically acceptable salt thereof.

2. A composition, comprising the compound of claim 1.

3. A composition, comprising the compound of claim 1 and a pharmaceutically acceptable carrier.

4. A method of treating a cancer or tumor in a subject in need thereof, comprising administering a therapeutically effective amount of a histone deacetylase 7 (HDAC-7) inhibitor to the subject.

5. The method of claim 4, wherein the HDAC-7 inhibitor comprises one or more of the compound of claim 1, or the composition of claim 2 or 3.

6. The method of claim 4, wherein the HDAC-7 inhibitor comprises one or more of TMP269, trichostatin A, vorinostat, or a pharmaceutically acceptable salt thereof.

7. The method of claim 4, wherein the HDAC-7 inhibitor comprises one or more siRNA or a pharmaceutically acceptable salt thereof, or a pharmaceutical composition comprising one or more of the siRNA or the pharmaceutically acceptable salt thereof.

8. The method of claim 7, wherein the siRNA comprises SEQ ID NO: 1, SEQ ID NO: 2, SEQ ID NO: 3, or SEQ ID NO: 4.

9. The method of claim 4, wherein the cancer or tumor comprises glioblastoma multiforme.

10. The method of claim 9, wherein the HDAC-7 inhibitor is administered before, after or concurrently with surgical removal of at least part of the cancer or tumor.

11. The method of claim 9, wherein the HDAC-7 inhibitor is administered in an amount sufficient to cause one or more of the following: a decrease in the cancer or tumor growth, a decrease in the cancer or tumor cell proliferation, an increase in apoptosis of one or more cells of the cancer or tumor, inhibition of metastatic dissemination of the glioblastoma multiforme, improvement in subject survival, reduced ability of cells of the cancer or tumor to form colonies, or reduced ability of cells of the cancer or tumor to migrate.

12. The method of claim 4, wherein the HDAC-7 inhibitor is administered before, after, or concurrently with administration of an antibody therapy.

13. The method of claim 4, wherein the HDAC-7 inhibitor is administered before, after, or concurrently with administration of a radiation therapy.

14. A pharmaceutical composition for treating glioblastoma multiforme, comprising one or more of: 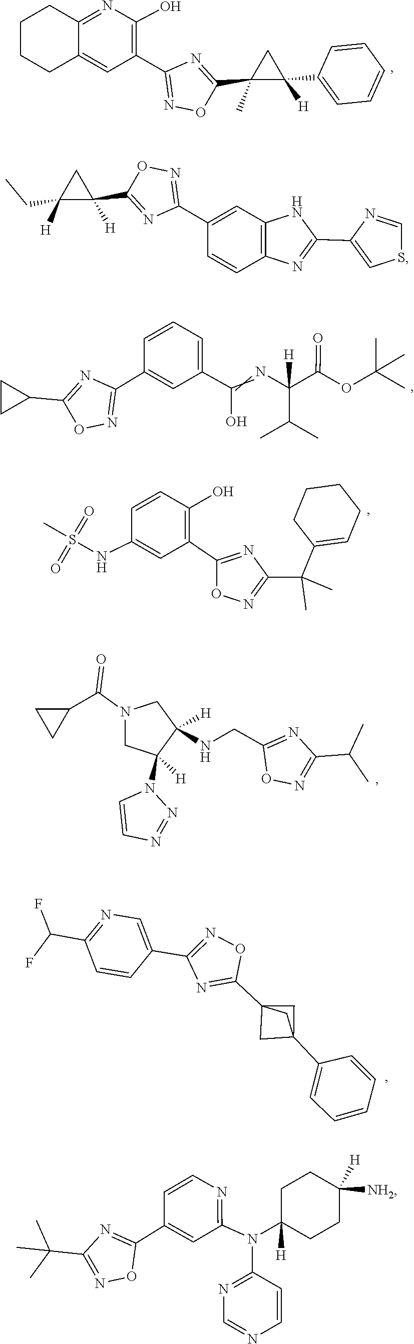 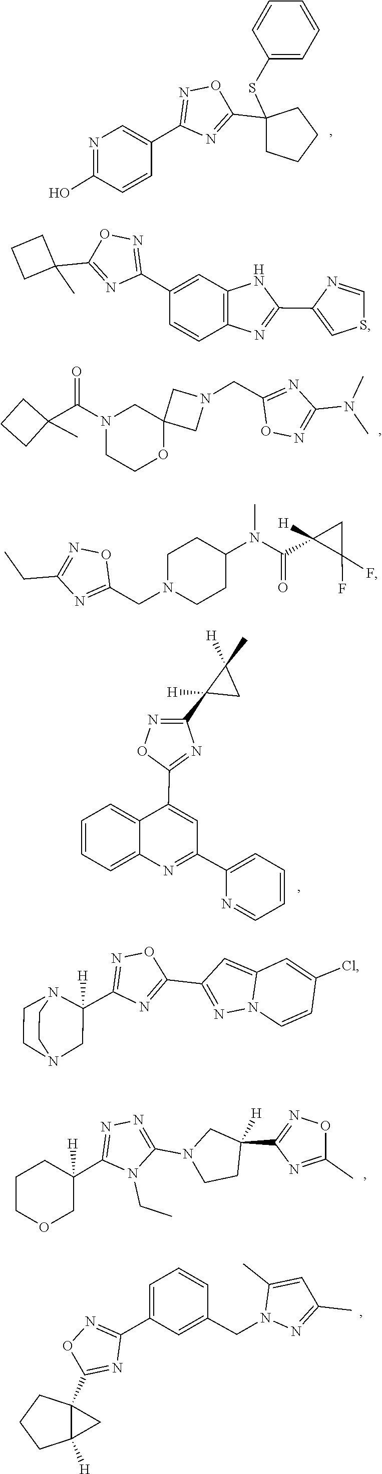 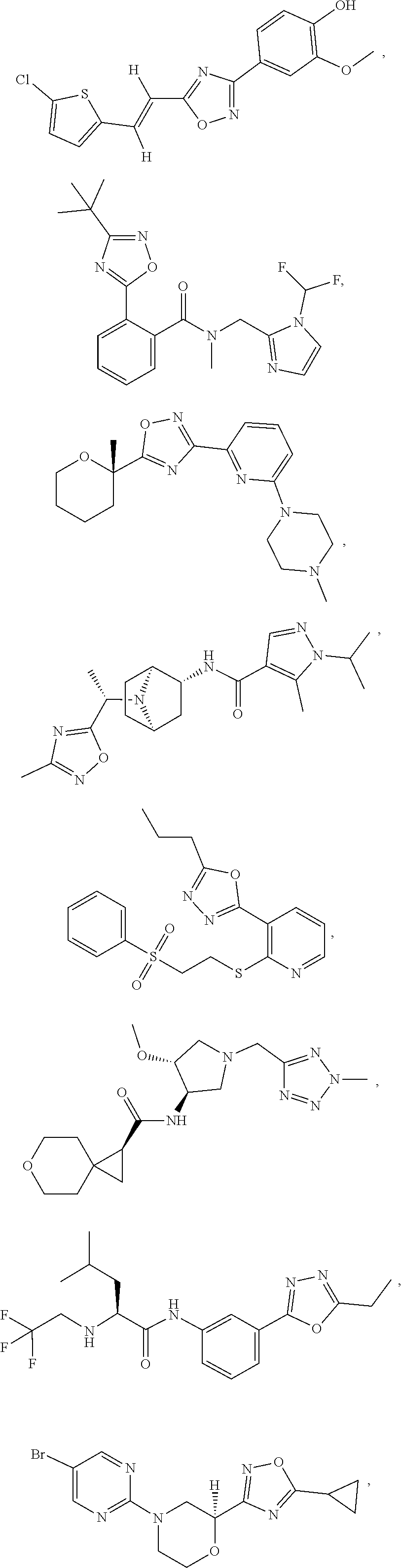 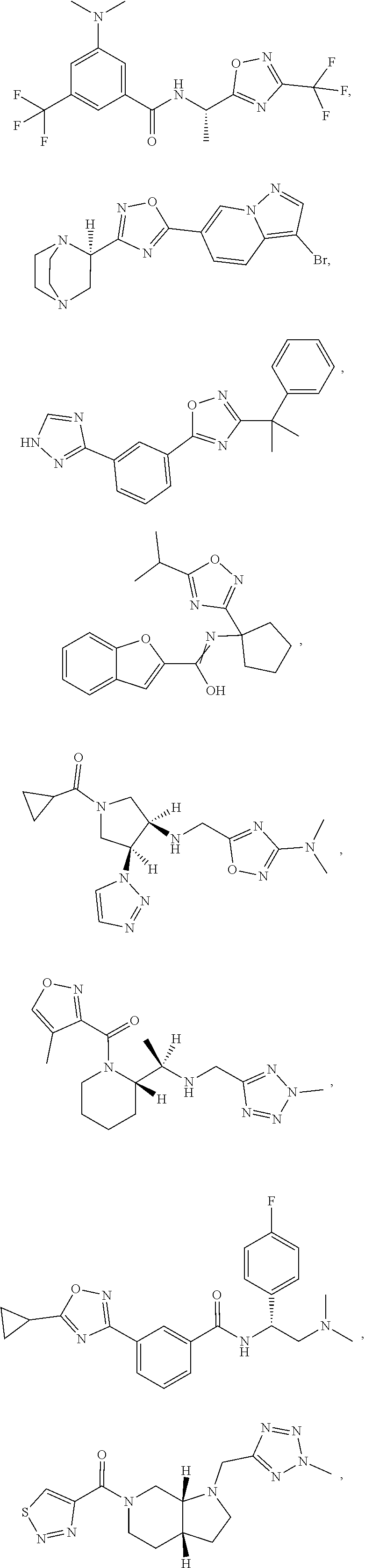 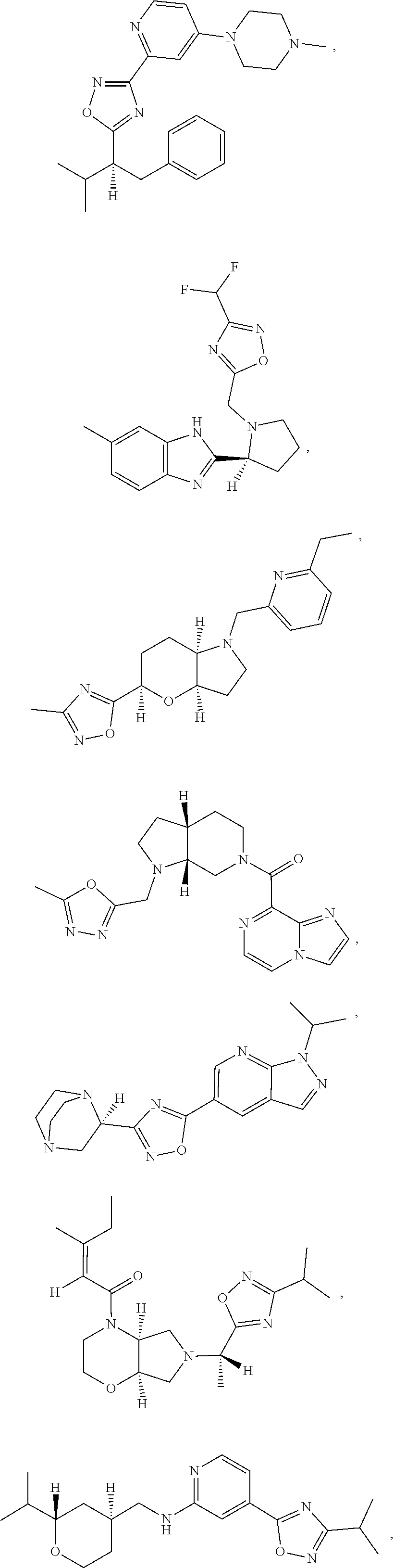 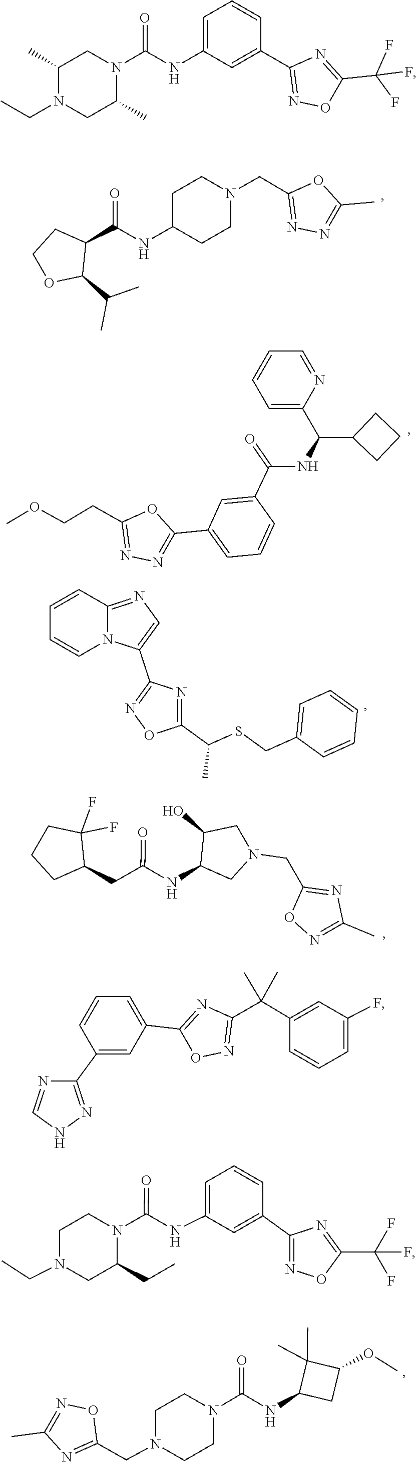 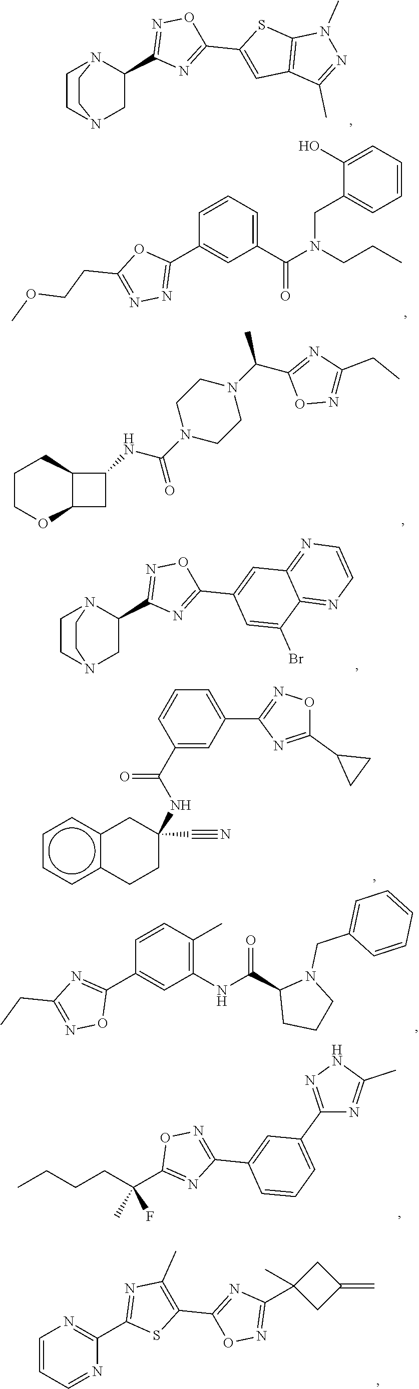 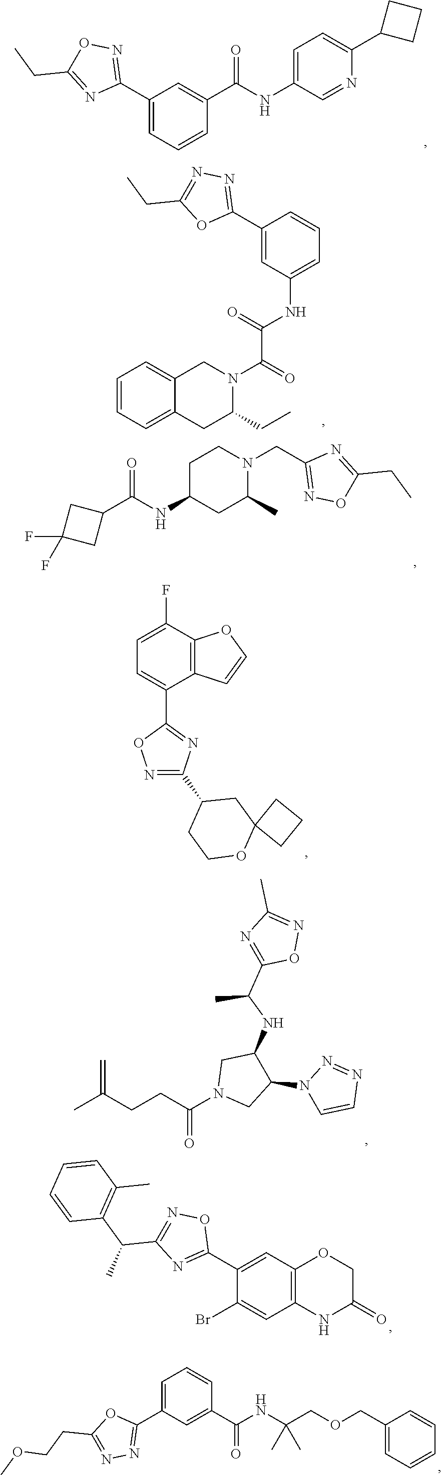 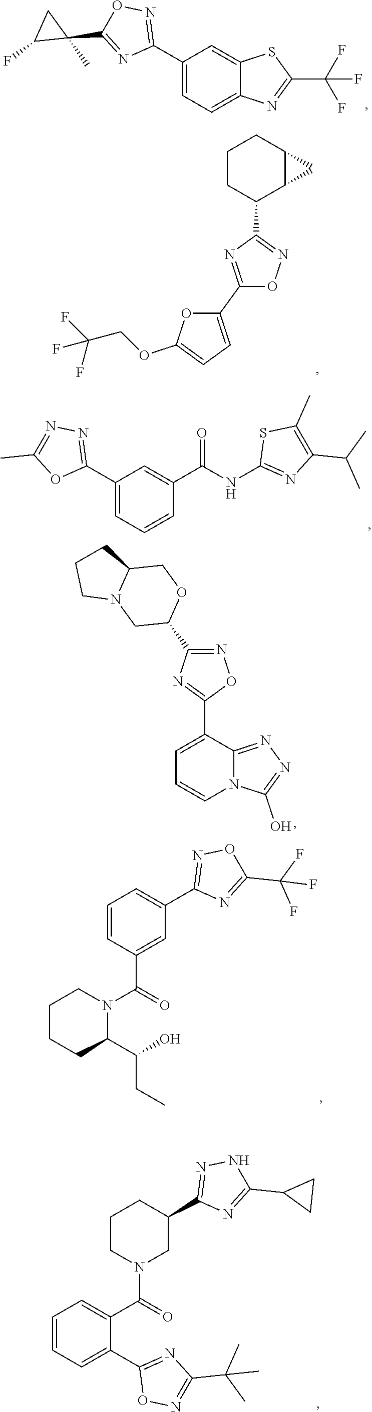 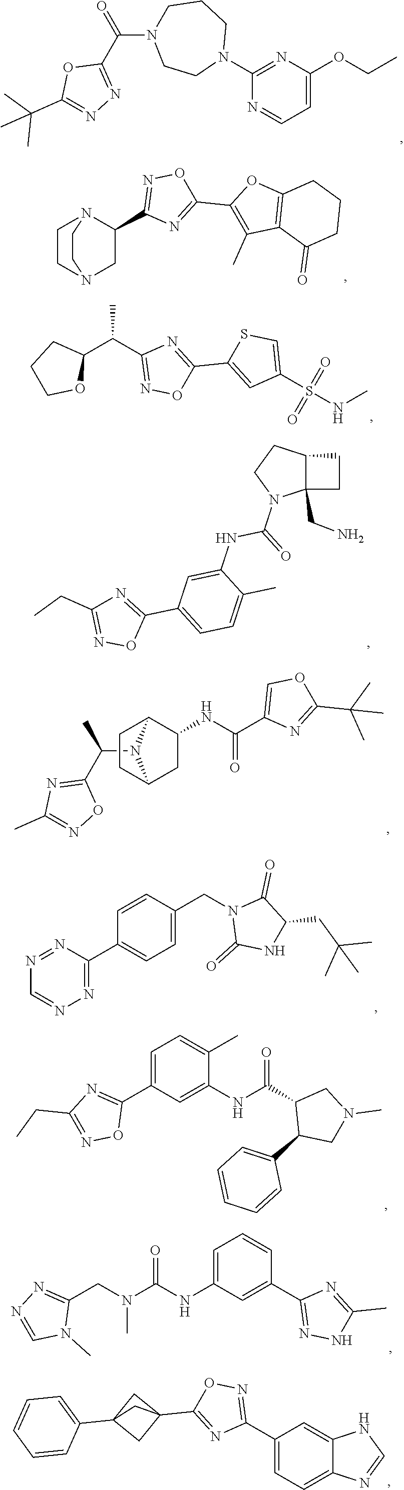 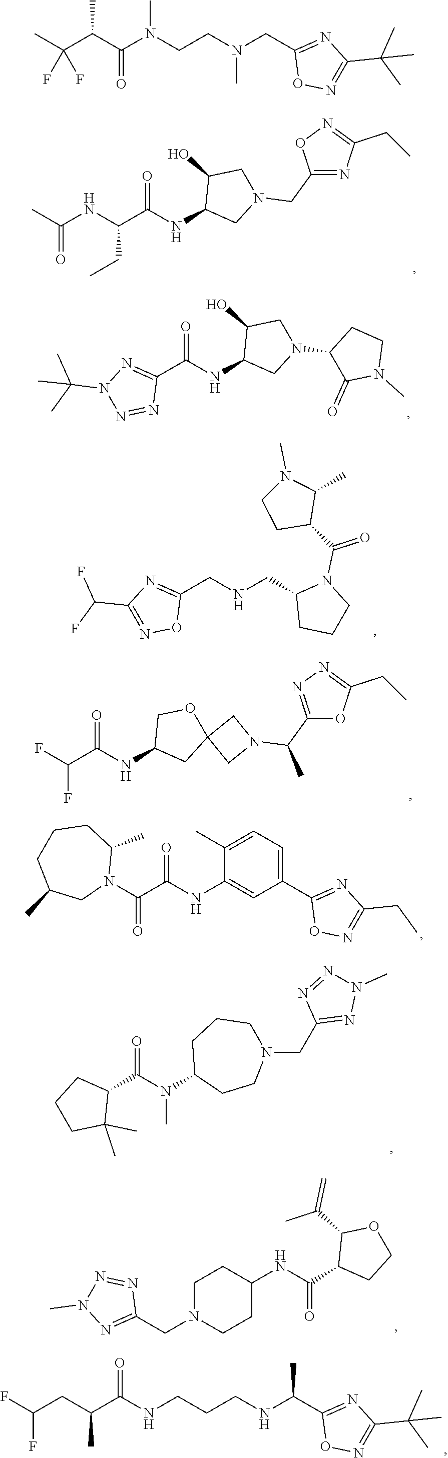 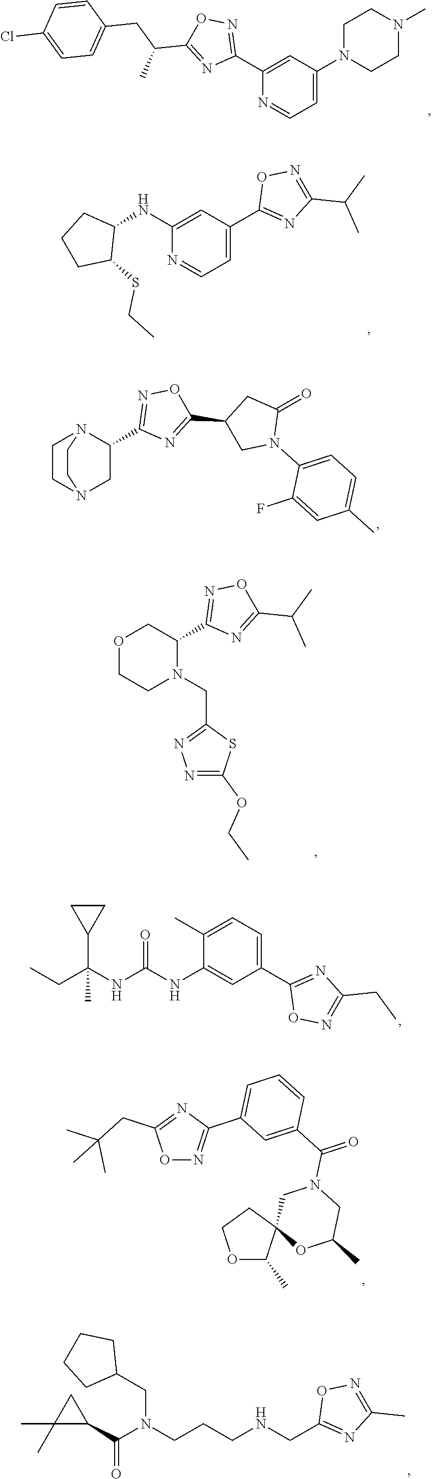 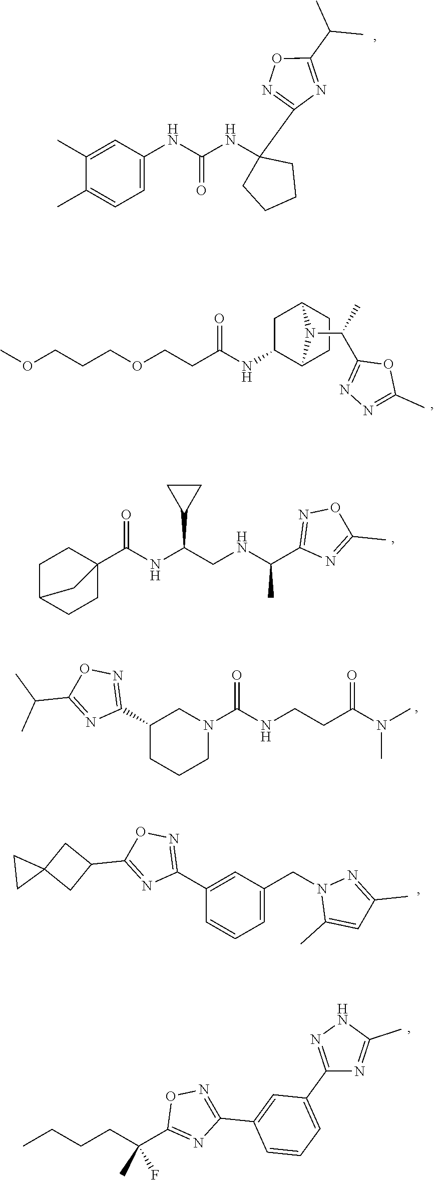 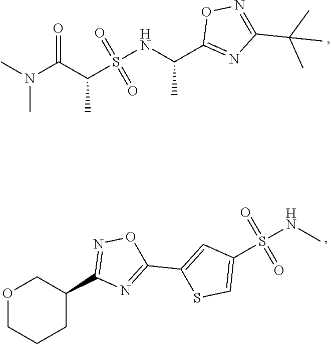 [SMILES: C[C@H](NS(=O)(=O)[C@H](C)C(=O)N(C)C)c1nc(C(C)(C)C)no1 ; CNS(=O)(=O)c1csc(-c2nc([C@H]3CCCOC3)no2)c1]
or a pharmaceutically acceptable salt thereof.

15. A pharmaceutical composition for treating glioblastoma multiforme, comprising one or more of: an siRNA, or a pharmaceutically acceptable salt thereof, wherein the siRNA comprises SEQ ID NO:1, SEQ ID NO:2, SEQ ID NO:3, or SEQ ID NO:4; TMP269 or a pharmaceutically acceptable salt thereof; trichostatin A or a pharmaceutically acceptable salt thereof; or vorinostat or a pharmaceutically acceptable salt thereof.


SEQUENCE LISTING (4 sequences)

SEQ ID NO: 1 - RNA, 19 nt, synthetic construct
  source 1..19: mol_type = other RNA
gacaagagcaagcgaagtg

SEQ ID NO: 2 - RNA, 19 nt, synthetic construct
  source 1..19: mol_type = other RNA
gcagataccctcggctgaa

SEQ ID NO: 3 - RNA, 19 nt, synthetic construct
  source 1..19: mol_type = other RNA
ggtgagggcttcaatgtca

SEQ ID NO: 4 - RNA, 19 nt, synthetic construct
  source 1..19: mol_type = other RNA
tggctgctcttctgggtaa